(19) United States
(12) Patent Application Publication
(10) Pub. No.: US 2014/0065162 A1
(43) Pub. Date: Mar. 6, 2014

(54) Compositions and Methods for Treating Brain Tumors

(71) Applicant: FibroGen, Inc., (US)

(72) Inventors: Kenneth E. Lipson, San Mateo, CA (US); Peter Ernst Huber, Heidelberg (DE)

(73) Assignee: FibroGen, Inc., San Francisco, CA (US)

(21) Appl. No.: 13/672,600

(22) Filed: Nov. 8, 2012

Related U.S. Application Data
(60) Provisional application No. 61/557,115, filed on Nov. 8, 2011.

(51) Int. Cl. A61K 39/395; A61K 45/06; A61K 31/713

(52) U.S. Cl. CPC A61K 39/3955; A61K 31/713; A61K 45/06
USPC 424/158.1; 514/44 A; 424/649

(57) ABSTRACT

The present invention relates to methods and medicaments useful for the treatment of brain tumors by administering anti-CTGF agents, particularly anti-CTGF antibodies. Methods and medicaments are provided for reducing tumor cell proliferation and tumor growth, reducing tumor vascularity, inhibiting tumor cell invasion, improving tumor surgical margins and prolonging survival of patients with brain tumors.

This application claims the benefit under 35 U.S.C. 119(e) of U.S. provisional application Ser. No. 61/557,115, filed on 8 Nov. 2011, that is incorporated by reference herein in its entirety.

### FIELD OF THE INVENTION

The present invention relates to methods and medicaments useful for the treatment of brain tumors. Methods and medicaments are provided for reducing tumor cell proliferation and tumor growth, reducing tumor vascularity, inhibiting tumor cell invasion, improving tumor margins and prolonging survival of patients with brain tumors.

### BACKGROUND OF THE INVENTION

Brain tumors may originate in the central nervous system (CNS) i.e., primary tumors, or they may be metastases from tumors in other organs or tissues. Malignant gliomas are the most prevalent type of primary tumors of the CNS and while they do not generally metastasize to other organs, malignant gliomas spread aggressively and often diffusely into normal brain tissue. Malignant gliomas encompass a family of primary CNS tumors including glioblastoma, astrocytoma, oligodendroglioma, ependymoma and pilocystic astrocytoma. Of special interest is glioblastoma multiforme (GBM), a form of malignant glioma that is particularly aggressive and invasive where standard therapy produces a median progression-free survival of 6.9 months, and a median overall survival of 14.6 months (Stupp et al, N Engl J Med 2005; 352: 987-96).

Approximately 40% of intracranial neoplasms are metastatic. Chemotherapy-based treatment is frequently ineffective as the metastases usually comprise drug resistant cancer cells and the blood brain barrier hinders treatment by limiting drug exposure. The mean survival for patients with small-cell lung cancer, breast cancer or melanoma brain metastases treated with chemotherapy alone is about 3.2-8 months. The addition of whole-brain radiation therapy (WBRT) provides a minimal extension of survival rates to about 3.5-13 months.

Given the dismal prognosis of patients with primary or metastatic brain tumors, a pressing and unmet medical need remains for improved treatments of brain tumors. The present invention addresses this unmet medical need by providing treatment methods and agents that inhibit tumor cell proliferation and tumor growth, decrease tumor microvascularization; impede tumor cell motility and decrease tumor cell invasiveness. Further, the methods and therapeutic agents of the invention extend patient survival.

### SUMMARY OF THE INVENTION

In one aspect of the invention, a method is provided for treating a brain tumor in a subject comprising administering to the subject in need thereof an effective amount of an anti-CTGF agent, thereby treating the brain tumor. In some embodiments, the brain tumor is a glioma. In further embodiments, the glioma is an astrocytoma or a glioblastoma. In additional embodiments, the brain tumor is a primary tumor. In other embodiments, the brain tumor is a metastasis. In further embodiments, the brain tumor is a recurrent tumor. In other embodiments, the brain tumor is not substantially resectable.

In some embodiments, the anti-CTGF agent is an anti-CTGF antibody, antibody fragment or antibody mimetic. In further embodiments, the anti-CTGF agent is an anti-CTGF antibody. In certain embodiments, the anti-CTGF antibody is identical to CLN-1. In other embodiments, the anti-CTGF antibody binds to the same epitope as CLN-1.

In other embodiments, the anti-CTGF agent is an anti-CTGF oligonucleotide. In further embodiments, the anti-CTGF oligonucleotide is an antisense oligonucleotide, siRNA, shRNA or miRNA.

In some embodiments, the anti-CTGF agent is administered as a neoadjuvant. In further embodiments, the anti-CTGF agent is administered with an additional therapeutic modality selected from the group consisting of chemotherapy, radiotherapy, immunotherapy and surgery. In other embodiments, chemotherapy comprises the administration of temozolmide, procarbazine, lomustine, vincristine, cisplatin, carmustine, carboplatin or methotrexate.

In some embodiments, administration of an anti-CTGF agent prolongs the survival of the subject. In further embodiments, the prolongation of survival is the prolongation of disease-free survival, progression-free survival or overall survival. In other embodiments, the prolongation of survival is at least 2 weeks, at least 1 month, at least 2 months, at least 3 months, at least 4 months, at least 5 months, at least 6 months, at least 8 months, at least 10 months, at least 12 months, at least 15 months, at least 18 months or at least 24 months longer compared to a control group or a historical control.

In some embodiments, treating a brain tumor comprises decreasing brain tumor cell invasiveness. In particular embodiments, the decrease in brain tumor cell invasiveness is radiation-induced invasiveness.

In some embodiments, treating a brain tumor comprises decreasing brain tumor cell proliferation, decreasing brain tumor vascularity, decreasing deposition of brain tumor extracellular matrix, decreasing intracellular levels of CTGF in brain tumor cells or reactive astrocytes, decreasing reactive astrocyte induction or decreasing the intracellular level of a stem cell marker in brain tumor cells.

In some embodiments, treating a brain tumor comprises improving the tumor surgical margin or increasing brain tumor cell differentiation.

### BRIEF DESCRIPTION OF THE DRAWINGS

FIGS. 1A and 1B illustrate the effect of an anti-CTGF antibody (CLN-1), irradiation (RT) or the combined treatment (CLN-1 and RT) on the in vitro migratory ability (motility) of U87MG and GBM cancer stem-like cells (CSLCs), NMA-23. Less U87MG and NMA-23 cells are apparent in the respective anti-CTGF antibody, irradiated and combined treatment wells compared to control. Fields are 20× magnification, cells were stained a Diff-Quik staining kit (Andwin Scientific, Schaumburg, Ill.).

FIGS. 2A and 2B demonstrate, respectively, the quantitative change in migratory ability (motility) of U87MG and NMA-23 tumor cells following treatment with an anti-CTGF antibody (CLN-1), irradiation (RT) or the combined treatment (CLN-1 and RT). FIG. 2A. Irradiation (4 Gy) did not significantly inhibit the relative number of U87MG cells that migrated compared to control (94.46±6.64% of control, p-value>0.05) Treatment with an anti-CTGF antibody, however, significantly inhibited the migration rate compared to control (44.60±5.75%, p-value<0.05), as did the combined treatment of anti-CTGF antibody and irradiation (60.53±1.44%, p-value<0.05). FIG. 2B. Similarly, irradiation did not significantly inhibit the relative number of NMA-23 cells that migrated compared to control (97.77±8.34%, p-value>0.05). Treatment with the anti-CTGF antibody, again, significantly inhibited migration of NMA-23 cells (71.91±5.52%, p-value<0.05) as did the combined treatment of anti-CTGF antibody and irradiation (68.06±2.95%, p-value<0.05). Columns=mean percentage; bars=SD; *=P<0.05 vs. control; #=P>0.05 vs. control; **=P<0.05 vs. control and irradiation.

FIGS. 3A and 3B illustrate, respectively, the decrease in surviving fraction of U87MG and T98G cells after treatment with an anti-CTGF antibody (CLN-1 irradiation (RT) or the combined treatment (CLN-1 and RT). FIG. 3A. Treatment of U87MG cells with the anti-CTGF antibody (30 μg/ml) significantly decreased the number of clones compared to control (66.95±3.11%, p-value<0.05). Irradiation (4 Gy) significantly decreased the number of clones compared to control (11.86±0.98%, p-value<0.05). The combined modalities significantly decreased the number of clones compared to control (6.89±2.13%, p-value<0.05). FIG. 3B. Treatment of T98G cells with the anti-CTGF antibody (30 μg/ml) significantly decreased the number of clones compared to control (71.43±10.04%, p-value<0.05). Irradiation significantly decreased the number of clones compared to control (37.37≦2.32%, p-value<0.05). The combined modalities significantly decreased the number of clones compared to control (25.86±1.98%, p-value<0.05). Columns=mean percentage; bars=SD; *=P<0.05 vs. control; **=P<0.05 vs. control and irradiation.

FIGS. 4A and 4B illustrate, respectively, the decrease in proliferation rate of U87MG and T98G glioblastoma cells observed following treatment with an anti-CTGF antibody (CLN-1), irradiation (RT) or the combined treatment (CLN-1 and RT). FIG. 4A. Treatment with the anti-CTGF antibody (30 μg/ml) significantly decreased U87MG cell number compared to control (59.24±14.05%, p-value<0.05). Irradiation (4 Gy) significantly decreased cell number compared to control (57.76±2.34%, p-value<0.05). The combined modalities (CLN-1, 30 μg/ml and 4 Gy irradiation) significantly decreased the cell number compared to control (45.21±6.46%, p-value<0.05). FIG. 4B. Treatment with the anti-CTGF antibody (30 μg/ml) significantly decreased T98G cells compared to control (58.65±6.31%, p-value<0.05). Irradiation (4 Gy) significantly decreased cell number compared to control (57.93±6.35%, p-value<0.05). The combined modalities (CLN-1, 30 μg/ml and 4 Gy irradiation) significantly decreased the cell number compared to control (36.42±0.36%, p-value<0.05). Columns=mean percentage; bars=SD; *=P<0.05 vs. control; **=P<0.05 vs. control and irradiation.

FIG. 5 shows representative fields from a clonogenic assay of GBM CSLCs (NMA-23, 10×). These cells form neurospheres in culture, a phenomenon that is associated with worse clinical outcomes. Treatment with anti-CTGF antibody (CLN-1, 30 μg/ml), irradiation (RT, 4 Gy), or the combination (CLN-1 and RT) decreased neurosphere formation.

FIG. 6 illustrates the decrease in the relative number of NMA-23 neurospheres observed in a clonogenic assay after treatment with an anti-CTGF antibody (CLN-1, 30 μg/ml), irradiation (RT, 4 Gy) or the combination (CLN-1 and RT). Neurospheres were counted 7 days after exposure to various treatments. Treatment with the anti-CTGF antibody significantly decreased neurosphere formation compared to control (60.52±12.53%, p-value<0.05). Irradiation significantly decreased neurosphere formation compared to control (30.62±2.52%, p-value<0.05). The combined treatment significantly decreased neurosphere formation compared to control (21.58±1.38%, p-value<0.05). Columns=mean percentage; bars=SD; *=P<0.05 vs. control; **=P<0.05 vs. control and irradiation.

FIG. 7 illustrates the decrease in the relative number of NMA-23 cells observed following treatment with an anti-CTGF antibody (CLN-1, 30 μg/ml), irradiation (RT, 4 Gy) or the combination (CLN-1 and RT). Cells were counted 72 h after exposure to various treatments. Treatment with the anti-CTGF antibody significantly decreased the number of NMA-23 cells compared to control (81.92±7.09% p-value<0.05). Irradiation significantly decreased the number of NMA-23 cells compared to control (81.21±9.11%, p-value<0.05). The combined treatment of anti-CTGF antibody and irradiation significantly decreased the cell number compared to control (52.64±11.51%, p-value<0.05). Columns=mean percentage; bars=SD; *=P<0.05 vs. control; **=P<0.05 vs. control and irradiation.

FIG. 8 illustrates the decrease in self-renewal capacity after treatment with an anti-CTGF antibody (CLN-1, 30 μg/ml), irradiation (RT, 4 Gy) or the combined treatment (CLN-1 and RT). Following treatment, limiting dilution assays were performed. After 7 days in culture, the percentage of wells not containing neurospheres for each cell-plating density was calculated (y-axis) and plotted against the number of cells plated per well (x-axis).

FIGS. 9A and 9B show representative T-1 weighted, gadolinium-enhanced MRI images of SCID-beige mouse brains taken on day 13 and day 17 post-implantation of NMA-23 cells. Mice were treated with anti-CTGF antibody (CLN-1, 3 mg/kg i.p., starting on days 6, 8, 10 and 12 post-implantation); irradiation (RT, 7 Gy on day 6); or the combined treatment (CLN-1 and RT). By day 13 post-implantation, the tumors in the control mice appear larger and denser compared to tumors in treated mice. At day 17 post-implantation, the tumor of the control mouse continued to increase in size at a faster rate compared to the treated tumors.

FIG. 10 illustrates the delay in tumor growth achieved in SCID-beige mice with orthotopically implanted NMA-23 cells treated with an anti-CTGF antibody (CLN-1, 3 mg/kg i.p., on days 6, 8, 10 and 12 post-implantation), irradiation (RT, 7 Gy on day 6) or the combined treatment (CLN-1 and RT). N=8 animals in each group. Tumor volumes were estimated from MRI images obtained 13 and 17 days post-implantation. Tumor volumes were calculated using the formula: Volume=(0.5×length)×(width²). At day 13 post-implantation, differences were observed between the different treatments, but significance was not achieved. At day 17 post-implantation, the difference in tumor volume became significant with control tumors having a mean volume of 165.7±49.47 mm³, tumors treated with the anti-CTGF antibody had a mean volume of 93.83-23.27 mm³ (p-value<0.05), irradiated tumors had a mean volume of 84.53±15.89 mm³ (p-value<0.05), while the combined treatment tumors had a tumor volume of 31.83±15.15 mm³ (p-value<0.05). Significance is marked for tumor volumes of day 17: *=P<0.05 vs. control; **=P<0.05 vs. control and monotherapy.

FIG. 11 is a Kaplan—Meier survival curve of SCID-beige mice with orthotopically implanted NMA-23 cells treated with an anti-CTGF antibody (CLN-1, 3 mg/kg i.p., on days 6, 8, 10 and 12 post-implantation), irradiation (RT, 7 Gy on day 6 post-implantation) or the combined treatment (CLN-1 and RT). Control group mice survived for 17.89±0.45 days. Mice treated with the anti-CTGF antibody survived for 20.89-0.79 days (p-value<0.05, log-rank). Mice treated with irradiation survived for 22.13±0.72 days (p-value<0.05, log-rank). The combined treatment prolonged survival to 23.88±1.01 days (p-value<0.05, log-rank). Survival differences were calculated with the help of a log-rank test. *=P<0.05 for median survival time.

FIG. 12 illustrates the degree of tumor cell infiltration into the surrounding brain parenchyma for control and treated mice that were implanted orthotopically with GBM stem cells (NMA-23). Representative paraffin-embedded samples stained with HE (10×) show that untreated tumors displayed modest infiltration of the brain, frequently organized in perivascular cell clusters. Anti-CTGF antibody treatment (CLN-1, 3 mg/kg i.p., every 2 days starting 6 days post-implantation) prevented the invasion of brain parenchyma. In contrast, irradiated tumors (RT, 7 Gy, 6 days post-implantation) displayed highly diffuse infiltration of the surrounding tissue with a diffuse shape of the tumor rim and single invading cells present. The combined treatment (CLN-1 and RT) attenuated irradiation-induced invasion resulting in a more sharply defined tumor rim and decrease in single invading cells.

FIG. 13 illustrates the decrease in intracellular CTGF levels achieved with different tumor treatments. SCID-beige mice orthotopically implanted into the striata with NMA-23 cells were treated with an anti-CTGF antibody (CLN-1, 3 mg/kg i.p., every 2 days starting 6 days post-implantation), irradiation (RT, 7 Gy, 6 days post-implantation) or the combined treatment (CLN-1 and RT). Sections of paraffin-embedded tumor samples were stained for intracellular CTGF and then representative fields were counted for each treatment group. Tumor cells from untreated mice (Control) were 86.97±7.6% positive for CTGF. Tumor cells from anti-CTGF antibody treated mice (CLN-1) were 42.37±13.95% positive for CTGF, p-value<0.05. Tumor cells from irradiated mice (RT) were 84.11±7.59% positive for CTGF, p-value>0.05. Tumor cells subject to the combination treatment (CLN-1 and RT) had 42.99±11.0% CTGF positive cells, p-value<0.05. Columns=mean percentage; bars=SD; *=P<0.05 vs. control; #=P>0.05 vs. control; **=P<0.05 vs. control and irradiation.

FIG. 14 illustrates the decrease in the Ki-67, a marker for cellular proliferation, achieved with different tumor treatments. SCID-beige mice orthotopically implanted into the striata with NMA-23 cells were treated with an anti-CTGF antibody (CLN-1, 3 mg/kg i.p., every 2 days starting 6 days post-implantation), irradiation (RT, 7 Gy, 6 days post-implantation) or the combined treatment (CLN-1 and RT). Sections of paraffin-embedded tumor samples were stained for Ki-67 and then representative fields were counted for each treatment group. Untreated tumors showed 53.74±4.1% Ki-67 positive cells. Anti-CTGF antibody treated tumors showed 20.81±3.64% p-value<0.05. Irradiated tumors had 12.88±2.57% Ki-67 positive cells, p-value<0.05. The tumors treated with the combination therapy had 8.18±1.88% Ki-67 positive cells, p-value<0.05. Columns=mean percentage; bars=SD; *=P<0.05 vs. control; #=P>0.05 vs. control; **=P<0.05 vs. control and irradiation.

FIG. 15 illustrates the number of CD-31 positive endothelial cells, seen in tumor sections following different treatments and reflect the degree of tumor vascularization. GBMs are known to be highly vascularized. SCID-beige mice orthotopically implanted into the striata with GBM stem cells (NMA-23) were treated with an anti-CTGF antibody (CLN-1, 3 mg/kg i.p., every 2 days starting 6 days post-implantation), irradiation (RT, 7 Gy, 6 days post-implantation) or the combined treatment (CLN-1 and RT). Representative immunohistologically stained snap-frozen brain sections were used for quantitative evaluation of CD-31 positive blood vessels inside the tumors. Control section had 60.88±49.25 microvessels per field that was significantly decreased after treatment with an anti-CTGF antibody (44.0±4.47 vessels per field, p-value<0.05), RT (25.75±4.03 vessels per field, p-value<0.05) and the combined treatment (10.6±2.3 vessels per field, p-value<0.05). Columns=mean percentage; bars=SD; *=P<0.05 vs. control; #=P>0.05 vs. control; **=P<0.05 vs. control and irradiation.

FIGS. 16A and 16B illustrate, respectively, the decrease in collagen 4 and collagen 5A1 deposition seen in tumors following different treatments. SCID-beige mice orthotopically implanted into the striata with NMA-23 cells were treated with an anti-CTGF antibody (CLN-1, 3 mg/kg i.p., every 2 days starting 6 days post-implantation), irradiation (RT, 7 Gy, 6 days post-implantation) or the combined treatment (CLN-1 and RT). Snap frozen, immunohistochemically stained tissue sections were examined to quantitate the percentage of positive stained area visible in each field. FIG. 16A shows that the control tumors had 28.28±4.84% of the field staining positive for collagen IV. Treatment with the anti-CTGF antibody significantly decreased the percentage of collagen IV positive stained field to 10.17±4.12% (p-value<0.05). Irradiation decreased the percentage of positive stained collagen IV field to 20.53±5.44 (not significant, p-value>0.5). The combined treatment significantly decreased the percentage of positive field to 8.23±2.01 (p value<0.05). Columns=mean percentage; bars=SD; *=P<0.05 vs. control; #=P>0.05 vs. control; **=P<0.05 vs. control and irradiation. Similarly, FIG. 16B shows that the percentage of positive stained tumor tissue for collagen VA1 in control tumors was 21.34±2.06%. Treatment with the anti-CTGF antibody significantly decreased the percentage of collagen VA1 field to 5.6±1.52%, p-value<0.05. Irradiation decreased the size of the collagen VA1 positive field to 13.99±2.92%, p-value<0.05. The combined therapy further decreased the percentage of collagen VA1 positive field to 2.8±0.54%, p-value<0.05. Columns=mean percentage; bars=SD; *=P<0.05 vs. control; #=P>0.05 vs. control; **=P<0.05 vs. control and irradiation.

FIG. 17 shows the decrease in the number of SOX-2 positive tumor cells following different treatments. SOX-2 is a tumor stem cell marker. SCID-beige mice orthotopically implanted into the striata with NMA-23 cells were treated with an anti-CTGF antibody (CLN-1, 3 mg/kg i.p., every 2 days starting 6 days post-implantation), irradiation (RT, 7 Gy, 6 days post-implantation) or the combined treatment (CLN-1 and RT). Snap frozen, immunohistochemically stained tissue sections were examined to quantitate the percentage of positive stained area visible in each field. In untreated tumors, 96% of the fields contained SOX-2-positive cells. Treatment with the anti-CTGF antibody (CLN-1) significantly decreased the percentage of SOX-2 positive cells in the fields to 43%. Irradiation had no impact on SOX-2 expression as about 96% of the tumor cells treated with irradiation expressed SOX-2. Combined treatment with an anti-CTGF antibody and irradiation resulted in about 38% of tumor cells expressing SOX-2.

### DESCRIPTION OF THE INVENTION

It is to be understood that the invention is not limited to the particular methodologies, protocols, cell lines, assays, and reagents described herein, as these may vary. It is also to be understood that the terminology used herein is intended to describe particular embodiments of the present invention, and is in no way intended to limit the scope of the present invention as set forth in the appended claims.

Each of the limitations of the invention can encompass various embodiments of the invention. It is, therefore, anticipated that each of the limitations of the invention involving any one element or combinations of elements can be included in each aspect of the invention. This invention is not limited in its application to the details set forth in the following description or illustrated in the drawings. The invention is capable of other embodiments and of being practiced or of being carried out in various ways.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications cited herein are incorporated herein by reference in their entirety for the purpose of describing and disclosing the methodologies, reagents, and tools reported in the publications that might be used in connection with the present invention. Nothing herein is to be construed as an admission that the present invention is not entitled to antedate such disclosure by virtue of prior invention.

The practice of the present invention will employ, unless otherwise indicated, conventional methods of chemistry, biochemistry, molecular biology, cell biology, genetics, immunology and pharmacology, within the skill of the art. Such techniques are explained fully in the literature. See, e.g., Gennaro, A. R., ed. (1990) Remington's Pharmaceutical Sciences, 18th ed., Mack Publishing Co.; Colowick, S. et al., eds., Methods In Enzymology, Academic Press, Inc.; Handbook of Experimental Immunology, Vols. I-IV (D. M. Weir and C. C. Blackwell, eds., 1986, Blackwell Scientific Publications); Maniatis, T. et al., eds. (1989) Molecular Cloning: A Laboratory Manual, 2nd edition, Vols. I-III, Cold Spring Harbor Laboratory Press; Ausubel, F. M. et al., eds. (1999) Short Protocols in Molecular Biology, 4th edition, John Wiley & Sons; Ream et al., eds. (1998) Molecular Biology Techniques: An Intensive Laboratory Course, Academic Press); PCR (Introduction to Biotechniques Series), 2nd ed. (Newton & Graham eds., 1997, Springer Verlag).

### DEFINITIONS

As used herein the term “about” refers to ±10%.

As used herein and in the appended claims, the singular form “a”, “an” and “the” include plural references unless the context clearly dictates otherwise. For example, a reference to “an anti-CTGF agent” includes a plurality of such agents; a reference to an “antibody” is a reference to one or more antibodies and to equivalents thereof known to those skilled in the art, and so forth.

As used herein, the terms “subject” and “patient” are used interchangeably to refer to an individual. In a preferred embodiment, said subject is a mammal, preferably primate, and more preferably a human being.

As used herein the term “brain tumor” refers to an abnormal growth of cells intracranially, i.e., within the brain that can be benign or malignant, including abnormal growth of cells that originate and comprise the brain i.e., neurons, glial cells, astrocytes, oligodendrocytes, ependymal cells, lymphatic tissue, blood vessels, cranial nerves, pituitary gland and pineal gland, as well as metastases from cancers that originate from other organs, e.g., breast or prostate cancer. Brain tumors that arise from brain tissues include gliomas and non-gliomas. Specific examples of gliomas include astrocytomas, oligodendrogliomas and ependymomas. Non-glioma brain tumors include benign tumors, such as pituitary adenomas and malignant tumors, such as medullblastomas, primary CNS lymphomas, and CNS germ cell tumors.

In some embodiments, the brain tumor to be treated is a benign brain tumor. In other embodiments, the brain tumor to be treated is a malignant brain tumor. In certain embodiments, the brain tumor is an astrocytoma, an oligodendroglioma, an oligoastrocytoma, a ganglioglioma, an ependymoma, a meningioma, a pituitary adenoma, a primitive neuroectodermal tumor, a medullblastoma, a primary CNS lymphoma, or a CNS germ cell tumor. In further embodiments, the brain tumor is an acoustic neuroma, an anaplastic astrocytoma or a meningioma. In other embodiments, the brain tumor is a brain stem glioma, a craniopharyngioma, an ependyoma, a juvenile pilocytic astrocytoma, a medulloblastoma, an optic nerve glioma, primitive neuroectodermal tumor, or a rhabdoid tumor. In certain embodiments, the brain tumor is a pediatric brain tumor.

In some embodiments, the brain tumor is a GBM. In further embodiments, the GBM is an astrocytic tumor that includes giant cell glioblastoma and gliosarcoma. While gliomas do not metastasize by the bloodstream, but they can spread via the cerebrospinal fluid and cause “drop metastases” to the spinal cord. In some embodiments, the treatment of gliomas that have spread to beyond their point of origin are contemplated.

Gliomas may be classified histologically as astrocytomas, oligodendrogliomas, or tumors with morphological features of both astrocytes and oligodendrocytes, termed oligoastrocytomas. Gliomas may be categorized by grade based on the morphologic appearance of the tumor cells and the probability of the degree of tumor growth rate and spread. (See Kleihues P et al. Brain Pathol. 1993: 3(3): 255-68) Grading is related to the presence of certain histological features such as high cellularity, cellular pleomorphism, mitotic activity, microvascular proliferation, and necrosis. For instance, grade II astrocytomas have cellular pleomorphism, but none of the other histological features listed above; grade III anaplastic astrocytomas display mitotic activity in addition to cellular pleomorphism; while grade IV glioblastomas exhibit all of the above histological features including microvascular proliferation and/or necrosis. Astrocytic tumors are graded as pilocytic astrocytoma, grade I; astrocytoma, grade II; anaplastic astrocytoma, grade III; and glioblastoma, grade IV. Oligodendrogliomas and oligoastrocytomas are graded as grade II or anaplastic, grade III. In some embodiments, the brain tumor to be treated is a glioma. In further embodiments, the glioma to be treated is a selected from the group consisting of astrocytoma grade I, astrocytoma grade II, astrocytoma grade III and astrocytoma grade IV.

Brain tumors derived from CNS cells or support structures can be classified as primary or recurrent. In some embodiments, the brain tumor to be treated is a primary tumor. In other embodiments, the brain tumor to be treated is a recurrent brain tumor.

In some embodiments, the brain tumors are metastatic tumors from cancers that originated in other organs. Multiple, large autopsy series suggest that, in order of decreasing frequency, lung, breast, melanoma, renal, and colon cancers are the most common primary tumors to metastasize to the brain. (See Posner J B. Management of brain metastases. Rev Neurol (Paris). 1992; 148(6-7):477-87; Wen P Y and Loeffler J S. Management of brain metastases. Oncology (Huntingt). July 1999; 13(7):941-54, 957-61) Other sources of brain metastases include prostate cancer, ovarian cancer, head and neck cancer, neuroblastoma, osteosarcoma and lymphoma, however, treatment is contemplated of any brain metastasis regardless of the originating organ or tissue.

As used herein, “connective tissue growth factor” and “CTGF” refer to a matricellular protein belonging to a family of proteins identified as CCN proteins. CTGF may also be referred to within the art as “hypertrophic chondrocyte-specific protein 24,” “insulin-like growth factor-binding protein,” and “CCN2.” The CCN family contains six distinct members CYR61 (CCN1), CTGF (CCN2), NOV (CCN3), WISP-1 (wnt-1 inducible secreted protein-1, CCN4), WISP-2 (CCN5) and WISP-3 (CCN6). (See, e.g., O'Brian et al. (1990) Mol Cell Biol 10:3569-3577; Joliot et al. (1992) Mol Cell Biol 12:10-21; Ryseck et al. (1991) Cell Growth and Diff 2:225-233; Simmons et al. (1989) Proc Natl Acad Sci USA 86:1178-1182; Pennica et al. (1998) Proc Natl Acad Sci USA, 95:14717-14722; and Zhang et al. (1998) Mol Cell Biol 18:6131-6141.) CCN proteins are characterized by conservation of 38 cysteine residues that constitute over 10% of the total amino acid content and give rise to a modular structure with N- and C-terminal domains. The modular structure of CTGF includes conserved motifs for insulin-like growth factor binding proteins (IGF-BP) and von Willebrand's factor (VWC) in the N-terminal domain, and thrombospondin (TSP1) and a cysteine-knot motif in the C-terminal domain. CTGF and the other CCN proteins have diverse biological properties and depending on the cellular context can stimulate cell proliferation, migration, adhesion, extracellular matrix (ECM) deposition and angiogenesis. Although the present invention demonstrates the role of CTGF in glioma migration and invasiveness and further demonstrates that agents that target/inhibit CTGF activity are beneficial in treating brain tumors, the invention specifically contemplates a similar role for other CCN family members, particularly Cyr61.

In the methods of the invention for treating a brain tumor, “treating” a brain tumor intends administering a therapeutic agent (e.g., anti-CTGF agent) to the subject in need thereof in order to achieve a beneficial effect on the brain tumor including changes in pathological features of the brain tumor, the alleviation of symptoms, and improvement in prognosis or outcome, including improvement in survival of a subject with a brain tumor. Treating a brain tumor may be effected by eradicating, reducing, or stabilizing the disease; reducing the symptoms, duration of symptoms, or need for medication, reducing the pathological features of a brain tumor, or prolonging patient survival.

The methods of the invention are accomplished by administering to a subject in need thereof a therapy comprising an effective amount of an anti-CTGF agent. As used herein, the terms “anti-connective tissue growth factor agent” or “anti-CTGF agent” refer to any agent, molecule, macromolecule, compound, or composition that specifically and directly inhibits or decreases the expression of the CTGF gene including inhibiting or decreasing CTGF mRNA expression or activity, or specifically and directly inhibiting or decreasing CTGF protein expression or activity. Anti-CTGF agents that are capable of specifically and directly inhibiting or decreasing the expression of the CTGF gene include anti-CTGF oligonucleotides comprising antisense oligonucleotides, siRNA, shRNA and miRNA. Anti-CTGF agents that are capable of specifically and directly inhibiting or decreasing CTGF activities include, without limitation, anti-CTGF antibodies, antigen-binding fragments derived from anti-CTGF antibodies, anti-CTGF antibody mimetics, and other CTGF binding polypeptides, peptides, oligonucleotides, and non-peptide small molecules that specifically bind to CTGF and block its interaction with cofactors, membrane-associated protein or extracellular matrix components, for example aptamers. Anti-CTGF agents, for example, antagonize or interfere with CTGF's binding to one or more co-factors, such as TGF-β or bone morphogenic protein 4 (BMP-4); membrane-associated proteins such as integrins, the tyrosine kinase receptor type A (TrkA) or low density lipoprotein receptor-related protein 1 (LRP1), or extracellular matrix components such as heparin sulfate proteoglycans or fibronectin. The anti-CTGF agents used in the methods of the invention exert their effects specifically and directly on the CTGF gene, CTGF mRNA or CTGF protein, rather than through a non-specific inhibitory mechanism, e.g., a non-specific protease inhibitor or non-specific transcription inhibitors, or an indirect inhibitory mechanism, e.g., an inhibitor of a component of an upstream or downstream signaling pathway for CTGF such as losartan, an angiotensin II receptor antagonist.

As used herein, the terms “effective amount” and “therapeutically effective amount” are synonymous and when used in the context of the methods of the invention, i.e., a method comprising the administration of an effective amount of an anti-CTGF agent, the terms refer to the amount of an anti-CTGF agent that produces a beneficial or therapeutic effect. In specific embodiments of the methods of the invention, treatment with an “effective amount” of an anti-CTGF agent refers to an amount of an anti-CTGF agent that is sufficient to achieve at least one or more of the following effects: (i) decrease tumor angiogenesis or tumor vascularization; (ii) decrease tumor cell invasiveness including radiation-induced invasiveness, as measured by a reduction in the number of invasive cells and/or the distance that brain tumor cells invade normal brain tissue; (iii) decrease tumor cell motility, including radiation-induced tumor cell motility; (iv) decrease tumor cell proliferation or mitotic index; (v) eradicate tumor or reduce tumor size, e.g., volume or diameter, (vi) inhibit the rate of tumor growth as measured by changes in tumor volume; (vii) stabilize tumor (i.e. the volume of the brain tumor does not change+25%); (viii) improve the tumor surgical margin; (ix) inhibit or decrease tumor metabolism; (x) decrease tumor perfusion, i.e., blood flow; (xi) inhibit or decrease the deposition of extracellular matrix within the tumor and/or in the surrounding tissue; (xii) stabilize or decrease peritumoral inflammation or edema in a subject; (xiii) decrease the concentration of CTGF, VEGF or other angiogenic or inflammatory mediators (e.g., cytokines or interleukins) in biological specimens e.g., resected tumor or brain tissue, plasma, serum, cerebrospinal fluid or urine; (xiv) decrease intracellular CTGF levels in tumor cells or in cells that surround tumor cells including reactive astrocytes; (xv) decrease the rate of tumor recurrence following surgical resection; (xvi) decrease or ameliorate the severity of one or more symptoms associated with a brain tumor, (xvii) decrease the duration of one or more symptoms associated with a brain tumor, (xviii) prevent or inhibit the recurrence of one or more symptoms associated with a brain tumor, (xix) decrease the dosage of medication required to control one or more symptoms associated with a brain tumor, i.e., seizures or nausea; (xx) increase the tumor progression-free survival rate of brain tumor patients (xxi) increase the overall survival rate of brain tumor patients; (xxii) increase the disease-free survival rate of brain tumor patients; (xxiii) decrease patient mortality; (xxiv) increase the number of patients in remission; (xxv) decrease the hospitalization length or decrease the hospitalization rate of brain tumor patients; (xxvi) enhance or improve the therapeutic efficacy of another therapeutic modality (synergize), e.g., radiotherapy, chemotherapy or immunotherapy; and/or (xxvii) improve the quality of life as assessed by methods well known in the art, e.g., a questionnaire.

In some embodiments, the methods of the invention comprising the administration of an effective amount of an anti-CTGF agent decreases the rate of tumor cell growth or proliferation, decreases brain tumor cell motility or invasiveness including radiation-induced motility or radiation-induced invasiveness, or decreases tumor cell metabolism. In further embodiments, the methods of the invention comprising the administration of an effective amount of an anti-CTGF agent decreases the rate of tumor cell growth or proliferation, decreases brain tumor cell motility or invasiveness including radiation-induced motility or radiation-induced invasiveness, or decreases the tumor cell metabolism by at least 10%, at least 15%, at least 20%, at least 25%, at least 30%, at least 35%, at least 40%, at least 50%, at least 60%, at least 70%, at least 80% or at least 90% compared to controls.

In some embodiments, the methods of the invention comprising the administration of an effective amount of an anti-CTGF agent increases brain tumor cell apoptosis and/or increases the sensitivity of brain tumor cells to chemotherapy agents, immunotherapy and other biologic agents, or radiation. In further embodiments, the methods of the invention comprising the administration of an effective amount of an anti-CTGF agent increases brain tumor cell apoptosis and/or increases the sensitivity of brain tumor cells to chemotherapy agents, immunotherapy and other biologic agents, or radiation by at least 10%, at least 15%, at least 20%, at least 25%, at least 30%, at least 35%, at least 40%, at least 50%, at least 60%, at least 70%, at least 80% or at least 90% compared to controls.

In some embodiments, the methods of the invention comprising the administration of an effective amount of an anti-CTGF agent decreases the degree of brain tumor vascularity. In further embodiments, the methods of the invention comprising the administration of an effective amount of an anti-CTGF agent decreases the degree of brain tumor vascularity by at least 10%, at least 15%, at least 20%, at least 25%, at least 30%, at least 35%, at least 40%, at least 50%, at least 60%, at least 70%, at least 80% or at least 90% compared to controls.

In some embodiments, the methods of the invention comprising the administration of an effective amount of an anti-CTGF agent decreases the deposition of extracellular matrix by tumor cells, normal brain cells or reactive astrocytes that surround brain tumor cells or brain tumors. In further embodiments, the methods of the invention comprising the administration of an effective amount of an anti-CTGF agent decreases the degree of deposition of extracellular matrix by at least 10%, at least 15%, at least 20%, at least 25%, at least 30%, at least 35%, at least 40%, at least 50%, at least 60%, at least 70%, at least 80% or at least 90% compared to controls.

In some embodiments, the methods of the invention comprising the administration of an effective amount of an anti-CTGF agent decreases the intracellular levels of CTGF in brain tumor cells, reactive astrocytes and in normal brain cells surrounding brain tumor cells. In further embodiments, the methods of the invention comprising the administration of an effective amount of an anti-CTGF agent decreases the intracellular levels of CTGF by at least 10%, at least 15%, at least 20%, at least 25%, at least 30%, at least 35%, at least 40%, at least 50%, at least 60%, at least 70%, at least 80% or at least 90% compared to controls.

In some embodiments, the methods of the invention comprising the administration of an effective amount of an anti-CTGF agent decreases the intracellular level of stem cell marker in brain tumor cells. In particular embodiments, the stem cell marker is Nestin or SOX-2. In further embodiments, the methods of the invention comprising the administration of an effective amount of an anti-CTGF agent decreases the intracellular level of stem cell marker in brain tumor cells by at least 10%, at least 15%, at least 20%, at least 25%, at least 30%, at least 35%, at least 40%, at least 50%, at least 60%, at least 70%, at least 80% or at least 90% compared to controls. In some embodiments, the methods of the invention comprising the administration of an effective amount of an anti-CTGF agent increases brain tumor cell differentiation.

In some embodiments, the methods of the invention comprising the administration of an effective amount of an anti-CTGF agent inhibits reactive astrocyte induction. In further embodiments, the methods of the invention comprising the administration of an effective amount of an anti-CTGF agent inhibits reactive astrocyte induction by at least 10%, at least 15%, at least 20%, at least 25%, at least 30%, at least 35%, at least 40%, at least 50%, at least 60%, at least 70%, at least 80% or at least 90% compared to controls.

### Antibodies

The term “antibody” is used in the broadest sense and specifically covers monoclonal antibodies (including full length monoclonal antibodies), polyclonal antibodies, multispecific antibodies (e.g., bispecific antibodies), antibody fragments so long as they exhibit the desired biological activity and antibody mimetics.

The term “monoclonal antibody” as used herein refers to an antibody obtained from a population of substantially homogeneous antibodies, i.e., the individual antibodies comprising the population are identical except for possible mutations, e.g., naturally occurring mutations, that may be present. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to decrease its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein, Nature, 256:495-97 (1975); Harlow et al., Antibodies: A Laboratory Manual, (Cold Spring Harbor Laboratory Press, 2nd ed. 1988); recombinant DNA methods (see, e.g., U.S. Pat. No. 4,816,567); phage-display technologies (see, e.g., Clackson et al., Nature, 352: 624-628 (1991); Marks et al., J Mol Biol 222: 581-597 (1992); and Lee et al., J. Immunol Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc Natl Acad Sci USA 90: 2551 (1993); U.S. Pat. Nos. 5,545,807; 5,545,806; 5,569,825; 5,625,126; 5,633,425; and 5,661,016.

Monoclonal antibodies specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass (see, e.g., U.S. Pat. No. 4,816,567; and Morrison et al., Proc Natl Acad Sci USA 81:6851-6855 (1984)).

“Humanized” forms of non-human (e.g., murine) antibodies are chimeric antibodies that contain minimal sequence derived from non-human immunoglobulin. In some embodiments, a humanized antibody is a human immunoglobulin (recipient antibody) in which residues from a one or more hypervariable regions (HVRs) of the recipient are replaced by residues from one or more HVRs of a non-human species (donor antibody) such as mouse, rat, rabbit, or nonhuman primate having the desired specificity, affinity, and/or capacity. For further details, see, e.g., Jones et al., Nature 321:522-525 (1986); Riechmann et al., Nature 332:323-329 (1988); and U.S. Pat. Nos. 6,982,321 and 7,087,409.

A “human antibody” is one which possesses an amino acid sequence which corresponds to that of an antibody produced by a human and/or has been made using any of the techniques for making human antibodies (see e.g., Hoogenboom and Winter, J Mol Biol, 227:381 (1991); Marks et al., J Mol Biol, 222:581 (1991); Boerner et al., J Immunol, 147(1):86-95 (1991); Li et al., Proc Natl Acad Sci USA, 103:3557-3562 (2006) and U.S. Pat. Nos. 6,075,181 and 6,150,584).

A “naked antibody” for the purposes herein is an antibody that is not conjugated to a cytotoxic moiety or radiolabel. In some embodiments, the anti-CTGF antibody is a naked antibody.

In some embodiments, the anti-CTGF agent is an antibody that binds specifically to CTGF. The anti-CTGF antibody may be specific for CTGF endogenous to the species of the subject to be treated or may be cross-reactive with CTGF from one or more other species. In some embodiments, the antibody for use in the present methods is obtained from the same species as the subject in need. In other embodiments, the antibody is a chimeric antibody wherein the constant domains are obtained from the same species as the subject in need and the variable domains are obtained from another species. For example, in treating a human subject the antibody for use in the present methods may be a chimeric antibody having constant domains that are human in origin and variable domains that are mouse in origin. In preferred embodiments, the antibody for use in the present methods binds specifically to the CTGF endogenous to the species of the subject in need. Thus, in certain embodiments, the antibody is a human or humanized antibody, particularly a monoclonal antibody, that specifically binds human CTGF, GenBank Accession No. NP_{—}001892, SEQ ID NO: 1.

Exemplary anti-CTGF antibodies for use in the methods of the present invention are described, e.g., in U.S. Pat. No. 5,408,040; PCT/US1998/016423; PCT/US1999/029652 and International Publication No. WO 99/33878. Preferably, the anti-CTGF antibody for use in the method is a monoclonal antibody. Preferably the antibody is a neutralizing antibody. In particular embodiments, the anti-CTGF antibody is an antibody described and claimed in U.S. Pat. Nos. 7,405,274 and 7,871,617. In some embodiments, the anti-CTGF antibody has the amino acid sequence of the antibody produced by the cell line identified by ATCC Accession No. PTA-6006. In other embodiments, the anti-CTGF antibody binds to CTGF competitively with an antibody produced by ATCC Accession No. PTA-6006. In further embodiments, the anti-CTGF antibody binds to the same epitope as the antibody produced by ATCC Accession No. PTA-6006. In specific embodiments, the antibody for use in the present methods is identical to CLN-1 or mAb1, as described in U.S. Pat. No. 7,405,274 and U.S. patent application Ser. No. 12/148,922, or an antibody substantially equivalent thereto or derived therefrom.

As referred to herein, the phrase “an antibody that specifically binds to CTGF” includes any antibody that binds to CTGF with high affinity. Affinity can be calculated from the following equation:

$$Affinity = {K}_{a} = \frac{[ Ab \cdot Ag ]}{[ Ab ] [ Ag ]} = \frac{1}{{K}_{d}}$$

where [Ab] is the concentration of the free antigen binding site on the antibody, [Ag] is the concentration of the free antigen, [Ab—Ag] is the concentration of occupied antigen binding sites, Ka is the association constant of the complex of antigen with antigen binding site, and Kd is the dissociation constant of the complex. A high-affinity antibody typically has an affinity at least on the order of 10⁸ M⁻¹, 10⁹ M⁻¹ or 10¹⁰ M⁻¹. In particular embodiments, an antibody for use in the present methods will have a binding affinity for CTGF between of 10⁸ M⁻¹ and 10¹⁰ M⁻¹, between 10⁸ M⁻¹ and 10⁹ M⁻¹ or between 10⁹ M⁻¹ and 10¹⁰ M⁻¹. In some embodiments the high-affinity antibody has an affinity of about 10⁸ M⁻¹, 10⁹ M⁻¹ or 10¹⁰ M⁻¹.

“Antibody fragments” comprise a functional fragment or portion of an intact antibody, preferably comprising an antigen binding region thereof. A functional fragment of an antibody will be a fragment with similar (not necessarily identical) specificity and affinity to the antibody which it is derived. Non-limiting examples of antibody fragments include Fab, F(ab′)₂, Fv fragments that can be produced through enzymatic digestion of whole antibodies, e.g., digestion with papain, to produce Fab fragments. Other non-limiting examples include engineered antibody fragments such as diabodies (Holliger P et al. Proc Natl Acad Sci USA. 1993, 90: 6444-6448); linear antibodies (Zapata et al. 1995 Protein Eng, 8(10):1057-1062); single-chain antibody molecules (Bird K D et al. Science, 1988, 242: 423-426); single domain antibodies, also known as nanobodies (Ghahoudi M A et al. FEBS Lett. 1997, 414: 521-526); domain antibodies (Ward E S et al. Nature. 1989, 341: 544-546); and multispecific antibodies formed from antibody fragments.

### Antibody Mimetics

Antibody mimetics are proteins, typically in the range of 3-25 kD that are designed to bind an antigen with high specificity and affinity like an antibody, but are structurally unrelated to antibodies. Frequently, antibody mimetics are based on a structural motif or scaffold that can be found as a single or repeated domain from a larger biomolecule. Examples of domain derived antibody mimetics included AdNectins that utilize the 10th fibronectin III domain (Lipov\check{s}ek D. Protein Eng Des Sel, 2010, 24:3-9); Affibodies that utilize the Z domain of staphylococcal protein A (Nord K et al. Nat Biotechnol. 1997, 15: 772-777) and DARPins that utilize the consensus ankyrin repeat domain (Amstutz P. Protein Eng Des Sel. 2006, 19:219-229. Alternatively, antibody mimetics can also be based on the entire structure of a smaller biomolecule, such as Anticalins that utilize the lipocalin structure (Beste G et al. Proc Natl Acad Sci USA. 1999, 5:1898-1903)

### Oligonucleotides

In some aspects, the present invention comprises synthetic oligonucleotides that decrease the expression of human CTGF mRNA. These anti-CTGF oligonucleotides include isolated nucleic acids, nucleic acid mimetics, and combinations thereof. Oligonucleotides of the invention comprise antisense oligonucleotides, ribozymes, external guide sequence (EGS) oligonucleotides (oligozymes) and inhibitory RNA (RNAi) including siRNA, miRNA (microRNA), and short hairpin RNA (shRNA). Oligonucleotides that decrease the expression of CTGF mRNA are useful for treating brain tumors, in particular, gliomas or metastases. In some embodiments, the anti-CTGF oligonucleotide is not a siRNA. In further embodiments, the anti-CTGF oligonucleotide is not an antisense oligonucleotide.

The terms “oligonucleotide” and “oligomeric nucleic acid” refer to oligomers or polymers of ribonucleic acid (RNA), deoxyribonucleic acid (DNA), mimetics or analogs of RNA or DNA, or combinations thereof, in either single- or double-stranded form. Oligonucleotides are molecules formed by the covalent linkage of two or more nucleotides or their analogs. Unless otherwise indicated, all nucleic acid sequences disclosed herein are expressed in the 5′-3′ direction. Additionally, unless otherwise indicated, a particular nucleic acid sequence in addition to explicitly indicating the disclosed sequence also implicitly encompasses conservatively modified variants thereof (e.g., degenerate codon substitutions), alleles, and complementary sequences.

The terms “complementary” and “complementarity” refer to conventional Watson-Crick base-pairing of nucleic acids. For example, in DNA complementarity, guanine forms a base pair with cytosine and adenine forms a base pair with thymine, whereas in RNA complementarity, guanine forms a base pair with cytosine, but adenine forms a base pair with uracil in place of thymine. An oligonucleotide is complementary to a RNA or DNA sequence when the nucleotides of the oligonucleotide are capable of forming hydrogen bonds with a sufficient number of nucleotides in the corresponding RNA or DNA sequence to allow the oligonucleotide to hybridize with the RNA or DNA sequence. In some embodiments, the oligonucleotides have perfect complementarity to human CTGF mRNA, i.e., no mismatches.

When used in the context of an oligonucleotide, “modified” or “modification” refers to an oligonucleotide that incorporates one or more unnatural (modified) sugar, nucleobase or internucleoside linkage. Modified oligonucleotides are structurally distinguishable, but functionally interchangeable with naturally occurring or synthetic unmodified oligonucleotides and usually have enhanced properties such as increased resistance to degradation by exonucleases and endonucleases, or increased binding affinity. In some embodiments, the anti-CTGF oligonucleotides are modified.

Unnatural covalent internucleoside linkages, i.e., modified backbones, include those linkages that retain a phosphorus atom in the backbone and also those that do not have a phosphorus atom in the backbone. Numerous phosphorous containing modified oligonucleotide backbones are known in the art and include, for example, phosphoramidites, phosphorodiamidate morpholinos, phosphorothioates, phosphorodithioates, phosphotriesters, aminoalkylphosphotri-esters, methyl and other alkyl phosphonates including 3′-alkylene phosphonates, 5′-alkylene phosphonates and chiral phosphonates, and phosphinates. In some embodiments, the modified oligonucleotide backbones that are without phosphorus atoms comprise short chain alkyl or cycloalkyl internucleoside linkages, mixed heteroatom and alkyl or cycloalkyl internucleoside linkages, or one or more short chain heteroatomic or heterocyclic internucleoside linkages. See Swayze E. and Bhat B. in Antisense Drug Technology Principles, Strategies, and Applications. 2nd Ed. CRC Press, Boca Rotan Fla., 2008 p. 144-182.

In further embodiments, the unnatural internucleoside linkages are uncharged and in others, the linkages are achiral. In some embodiments, the unnatural internucleoside linkages are uncharged and achiral, e.g., peptide nucleic acids (PNAs).

In some embodiments, the modified sugar moiety is a sugar other than ribose or deoxyribose. In certain embodiments, the sugar is arabinose, xylulose or hexose. In further embodiments, the sugar is substituted with one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. In some embodiments, the modifications include 2′-methoxy (2′-O—CH3), 2′-aminopropoxy (2′-OCH2CH2CH2NH2), 2′-allyl (2′-CH2-CH═CH2), 2′-O-allyl (2′-O—CH2-CH═CH2) and 2′-fluoro (2′-F). The 2′-modification may be in the arabino (up) position or ribo (down) position. Similar modifications may also be made at other positions on an oligonucleotide, particularly the 3′ position of the sugar on the 3′ terminal nucleotide or in 2′-5′ linked oligonucleotides and the 5′ position of 5′ terminal nucleotide.

In some embodiments, the modified sugar is conformationally restricted. In further embodiments, the conformational restriction is the result of the sugar possessing a bicyclic moiety. In still further embodiments, the bicyclic moiety links the 2′-oxygen and the 3′ or 4′-carbon atoms. In some embodiments the linkage is a methylene (—CH2-)n group bridging the 2′ oxygen atom and the 4′ carbon atom, wherein n is 1 or 2. This type of structural arrangement produces what are known as “locked nucleic acids” (LNAs). See Koshkin et al. Tetrahedron, 54, 3607-3630, 1998; and Singh et al., Chem. Commun, 455-456, 1998.

In some embodiments, the sugar is a sugar mimetic that is conformationally restricted resulting in a conformationally constrained monomer. In some embodiments, the sugar mimetic comprises a cyclohexyl ring that comprises one ring heteroatom and a bridge making the ring system bicyclic. See PCT/US2010/044549. In further embodiments, the oligonucleotides comprise at least one nucleotide that has a bicyclic sugar moiety or is otherwise conformationally restricted.

In some embodiments, the modified sugar moiety is a sugar mimetic that comprises a morpholino ring. In further embodiments, the phosphodiester internucleoside linkage is replaced with an uncharged phosphorodiamidate linkage. See Summerton, Antisense Nucleic Acid Drug Dev, 7: 187-195,1997.

In some embodiments, both the phosphate groups and the sugar moieties are replaced with a polyamide backbone comprised of repeating N-(2-aminoethyl)-glycine units to which the nucleobases are attached via methylene carbonyl linkers. These constructs are called peptide nucleic acids (PNAs). PNAs are achiral, uncharged and because of the peptide bonds, resistant to endo- and exonucleases. See Nielsen et al., Science, 1991, 254, 1497-1500 and U.S. Pat. No. 5,539,082.

Oligonucleotides useful in the methods of the invention include those comprising entirely or partially of naturally occurring nucleobases. Naturally occurring nucleobases include adenine, guanine, thymine, cytosine, uracil, 5-methylcytidine, pseudouridine, dihydrouridine, inosine, ribothymidine, 7-methylguanosine, hypoxanthine and xanthine.

Oligonucleotides further include those comprising entirely or partially of modified nucleobases (semi-synthetically or synthetically derived). Modified nucleobases include 5-methylcytosine (5-me-C), 5-hydroxymethyl cytosine, hypoxanthine, 2-aminoadenine, 2-methyladenine, 6-methyladenine, 2-propyladenine, N6-adenine, N6-isopentenyladenine, 2-methylthio-N6-isopentenyladenine, 2-methylguanine, 6-methylguanine, 2-propylguanine, 1-methylguanine, 7-methylguanine, 2,2-dimethylguanine, 2-thiouracil, 2-thiothymine and 2-thiocytosine, 5-fluorouracil, 5-bromouracil, 5-chlorouracil, 5-iodouracil, dihydrouracil, 5-methyl-2-thiouracil, 2-thiouracil, 4-thiouracil, 5-methyluracil, uracil-5-oxyacetic acid methylester, uracil-5-oxyacetic acid, 5-carboxymethylaminomethyl-2-thiouridine, hypoxanthine, xantine, 4-acetylcytosine, 5-(carboxyhydroxylmethyl) uracil, 5-methoxycarboxymethyluracil, 5-methoxyuracil, 5-methyl-2-thiouracil, 3-(3-amino-3-N-2-carboxypropyl) uracil, 5-carboxymethylaminomethyluracil, 5-methylaminomethyluracil, 5-methoxyaminomethyl-2-thiouracil, 5-propynyl uracil and cytosine and other alkynyl derivatives of pyrimidine bases, 6-azo uracil, cytosine and thymine, 5-uracil (pseudouracil), 4-thiouracil, 8-halo-adenine, 8-amino adenine, 8-thiol adenine, 8-thioalkyl adenine, 8-hydroxyl adenine, 5-halo particularly 5-bromo, 5-trifluoromethyl uracil, 3-methylcytosine, 5-methylcytosine, 5-trifluoromethyl cytosine, 7-methylguanine and 7-methyladenine, 2-F-adenine, 2-amino-adenine, 8-azaguanine and 8-azaadenine, 7-deazaguanine, 8-halo-guanine, 8-amino guanine, 8-thiol guanine, 8-thioalkyl guanine, 8-hydroxyl guanine, 7-deazaadenine, 3-deazaguanine, 3-deazaadenine, beta-D-galactosylqueosine, beta-D-mannosylqueosine, inosine, 1-methylinosine, 2,6-diaminopurine and queosine. Further modified nucleobases include tricyclic pyrimidines such as phenoxazine cytidine(1H-pyrimido[5,4-b][1,4]benzoxazin-2(3H)-one), and phenothiazine cytidine (1H-pyrimido[5,4-b][1,4]benzothiazin-2(3H)-one. See Herdewijn P, Antisense Nucleic Acid Drug Dev 10: 297-310, 2000; and Sanghvi Y S, et al. Nucleic Acids Res, 21: 3197-3203, 1993.

In some embodiments, at least one nucleoside, i.e., a joined base and sugar, in an oligonucleotide is modified, i.e., a nucleoside mimetic. In certain embodiments, the modified nucleoside comprises a tetrahydropyran nucleoside, wherein a substituted tetrahydropyran ring replaces the naturally occurring pentofuranose ring. See PCT/US2010/022759 and PCT/US2010/023397. In other embodiments, the nucleoside mimetic comprises a 5′-substituent and a 2′-substituent. See PCT/US2009/061913. In some embodiments, the nucleoside mimetic is a substituted α-L-bicyclic nucleoside. See PCT/US2009/058013. In additional embodiments, the nucleoside mimetic comprises a bicyclic sugar moiety. See PCT/US2009/039557. In further embodiments, the nucleoside mimetic comprises a bis modified bicyclic nucleoside. See PCT/US2009/066863. In certain embodiments, the nucleoside mimetic comprises a bicyclic cyclohexyl ring wherein one of the ring carbons is replaced with a heteroatom. See PCT/US2009/033373. In still further embodiments, a 3′ or 5′-terminal bicyclic nucleoside is attached covalently by a neutral internucleoside linkage to the oligonucleotide. See PCT/US2009/039438. In other embodiments, the nucleoside mimetic is a tricyclic nucleoside. See PCT/US2009/037686.

The oligonucleotides of the invention can contain any number of the modifications described herein. In some embodiments, at least 5% of the nucleotides in the oligonucleotides are modified. In other embodiments, at least 10%, 15%, 20%, 30%, 35%, 40%, 45%, 50%, 55%, 60%, 65%, 70%, 75%, 80%, 85%, 90%, 95% or 99% of the nucleotides in the oligonucleotides are modified. In further embodiments, 100% of the nucleotides in the oligonucleotides are modified.

The aforementioned modifications may be incorporated uniformly across an entire oligonucleotide, at specific regions or discrete locations within the oligonucleotide including at a single nucleotide. Incorporating these modifications can create chimeric or hybrid oligonucleotides wherein two or more chemically distinct areas exist, each made up of one or more nucleotides.

Oligonucleotides of the invention can be synthesized by any method known in the art, e.g., using enzymatic synthesis and/or chemical synthesis. The oligonucleotides can be synthesized in vitro (e.g., using enzymatic synthesis and chemical synthesis) or in vivo (using recombinant DNA technology well known in the art). In a preferred embodiment, chemical synthesis is used for modified polynucleotides. Chemical synthesis of linear oligonucleotides is well known in the art and can be achieved by solution or solid phase techniques. Preferably, synthesis is by solid phase methods. Automated, solid phase oligonucleotide synthesizers used to construct the oligonucleotides of the invention are available through various vendors including GE Healthcare Biosciences (Piscataway, N.J.).

Oligonucleotide synthesis protocols are well known in the art and can be found, e.g., in U.S. Pat. No. 5,830,653; WO 98/13526; Stec et al. 1984 J Am Chem Soc 106:6077; Stec et al. 1985 J Org. Chem. 50:3908; Stec et al. J Chromatog 1985. 326:263; LaPlanche et al. 1986. Nucl Acid Res 1986. 14:9081; Fasman G. D., 1989. Practical Handbook of Biochemistry and Molecular Biology. 1989. CRC Press, Boca Raton, Fla.; U.S. Pat. No. 5,013,830; U.S. Pat. No. 5,214,135; U.S. Pat. No. 5,525,719; Kawasaki et al. Med Chem, 199336:831; WO 92/03568; U.S. Pat. No. 5,276,019; and U.S. Pat. No. 5,264,423.

In some embodiments, the decrease in expression of CTGF mRNA by an anti-CTGF oligonucleotide comprises the interference in the function of the target CTGF DNA sequence (CTGF gene), typically resulting in decreased replication and/or transcription of the target CTGF DNA. In other embodiments, the decrease in expression of CTGF mRNA by an anti-CTGF oligonucleotide comprises the interference in function of CTGF RNA, typically resulting in impaired splicing of transcribed CTGF RNA (pre-mRNA) to yield mature mRNA species, decreased CTGF RNA stability, decreased translocation of the CTGF mRNA to the site of protein translation and impaired translation of protein from mature mRNA. In other embodiments, the decrease in expression of CTGF mRNA by an anti-CTGF oligonucleotide comprises the decrease in cellular CTGF mRNA number or cellular content of CTGF mRNA. In some embodiments, the decrease in expression of CTGF mRNA by an anti-CTGF oligonucleotide comprises the down-regulation or knockdown of CTGF gene expression. In other embodiments, the decrease in expression of CTGF mRNA by an anti-CTGF oligonucleotide comprises the decrease in CTGF protein expression or cellular CTGF protein content.

In some embodiments, the methods of the invention comprising the administration of an effective amount of an anti-CTGF oligonucleotide decreases CTGF mRNA transcription rate, cellular CTGF mRNA level, CTGF expression rate, cellular CTGF protein level or tumor interstitial CTGF protein level. In further embodiments, the methods of the invention comprising the administration of an effective amount of an anti-CTGF oligonucleotide decreases CTGF mRNA transcription rate, cellular CTGF mRNA level, CTGF expression rate, cellular CTGF protein level or tumor interstitial CTGF protein level by at least 5%, at least 10%, at least 15%, at least 20%, at least 25%, at least 30%, at least 35%, at least 40%, at least 45%, at least 50%, at least 60%, at least 70%, at least 80% or at least 90% compared to controls.

As used herein, the terms “antisense oligonucleotide” and “ASO” refer to an oligomeric nucleic acid that is capable of hybridizing with its complementary target nucleic acid sequence resulting in the impairment of the normal function of the target nucleic acid sequence. Antisense oligonucleotides that inhibit CTGF expression have been described and utilized to decrease CTGF expression in various cell types. (See, e.g., PCT/US1996/008140; PCT/US1999/026189; PCT/US1999/029652; PCT/US2002/038618; Kothapalli et al. (1997) Cell Growth Differ 8:61-68; Shimo et al. (1998) J Biochem (Tokyo) 124:130-140; Uchio et al. (2004) Wound Repair Regen 12:60-66; Guha et al. (2007) FASEB J 21:3355-3368; U.S. Pat. No. 6,358,741; U.S. Pat. No. 6,965,025; U.S. Pat. No. 7,462,602; U.S. Patent Application Publication No. 2008/0070856; U.S. Patent Application Publication No. 2008/0176964.)

Preferred antisense oligonucleotides for use in methods of the invention include the “potent” antisense oligonucleotides disclosed in U.S. patent application Ser. No. 13/546,799. In some embodiments, the potent antisense oligonucleotides comprise at least a 9, 10, 1, 12, 13, 14, 15, 16, 17, 18 or 19 nucleotide sequence portion of a nucleotide sequence selected from Table 1. In further embodiments, the potent antisense oligonucleotides consist of a nucleotide sequence selected from Table 1.

TABLE 1
Antisense Oligonucleotides

| 5′ | 3′ | | |
|---|---|---|---|
| Target | Target | | |
| Site | Site | Antisense Sequence | SEQ ID NO: |
| 567 | 586 | AGGCACGTGCACTGGTACTT | SEQ ID NO: 2 |
| 569 | 588 | CCAGGCACGTGCACTGGTAC | SEQ ID NO: 3 |
| 613 | 632 | ACGAACGTCCATGCTGCACA | SEQ ID NO: 4 |
| 684 | 703 | CACACCCACTCCTCGCAGCA | SEQ ID NO: 5 |
| 721 | 740 | GGCAGGCCCAACCACGGTTT | SEQ ID NO: 6 |
| 747 | 766 | GTGTCTTCCAGTCGGTAAGC | SEQ ID NO: 7 |
| 789 | 808 | ACCAGGCAGTTGGCTCTAAT | SEQ ID NO: 8 |
| 791 | 810 | GGACCAGGCAGTTGGCTCTA | SEQ ID NO: 9 |
| 793 | 812 | CTGGACCAGGCAGTTGGCTC | SEQ ID NO: 10 |
| 795 | 814 | GTCTGGACCAGGCAGTTGGC | SEQ ID NO: 11 |
| 859 | 878 | GTCATTGGTAACCCGGGTGG | SEQ ID NO: 12 |
| 861 | 880 | TTGTCATTGGTAACCCGGGT | SEQ ID NO: 13 |
| 893 | 912 | GGCGGCTCTGCTTCTCTAGC | SEQ ID NO: 14 |
| 904 | 923 | GACCATGCACAGGCGGCTCT | SEQ ID NO: 15 |
| 912 | 931 | CAAGGCCTGACCATGCACAG | SEQ ID NO: 16 |
| 916 | 935 | TTCGCAAGGCCTGACCATGC | SEQ ID NO: 17 |
| 935 | 954 | TGTTCTCTTCCAGGTCAGCT | SEQ ID NO: 18 |
| 946 | 965 | GCCCTTCTTAATGTTCTCTT | SEQ ID NO: 19 |
| 948 | 967 | TTGCCCTTCTTAATGTTCTC | SEQ ID NO: 20 |
| 950 | 969 | TTTTGCCCTTCTTAATGTTC | SEQ ID NO: 21 |
| 952 | 971 | CTTTTTGCCCTTCTTAATGT | SEQ ID NO: 22 |
| 954 | 973 | CACTTTTTGCCCTTCTTAAT | SEQ ID NO: 23 |
| 956 | 975 | TGCACTTTTTGCCCTTCTTA | SEQ ID NO: 24 |
| 958 | 977 | GATGCACTTTTTGCCCTTCT | SEQ ID NO: 25 |
| 960 | 979 | CGGATGCACTTTTTGCCCTT | SEQ ID NO: 26 |
| 962 | 981 | TACGGATGCACTTTTTGCCC | SEQ ID NO: 27 |
| 965 | 984 | GAGTACGGATGCACTTTTTG | SEQ ID NO: 28 |
| 967 | 986 | GGGAGTACGGATGCACTTTT | SEQ ID NO: 29 |
| 969 | 988 | TTGGGAGTACGGATGCACTT | SEQ ID NO: 30 |
| 981 | 1000 | GGCTTGGAGATTTTGGGAGT | SEQ ID NO: 31 |
| 983 | 1002 | TAGGCTTGGAGATTTTGGGA | SEQ ID NO: 32 |
| 989 | 1008 | ACTTGATAGGCTTGGAGATT | SEQ ID NO: 33 |
| 991 | 1010 | AAACTTGATAGGCTTGGAGA | SEQ ID NO: 34 |
| 1006 | 1025 | GCAGCCAGAAAGCTCAAACT | SEQ ID NO: 35 |
| 1008 | 1027 | GTGCAGCCAGAAAGCTCAAA | SEQ ID NO: 36 |
| 1011 | 1030 | CTGGTGCAGCCAGAAAGCTC | SEQ ID NO: 37 |
| 1013 | 1032 | TGCTGGTGCAGCCAGAAAGC | SEQ ID NO: 38 |
| 1015 | 1034 | CATGCTGGTGCAGCCAGAAA | SEQ ID NO: 39 |
| 1017 | 1036 | TTCATGCTGGTGCAGCCAGA | SEQ ID NO: 40 |
| 1019 | 1038 | TCTTCATGCTGGTGCAGCCA | SEQ ID NO: 41 |
| 1021 | 1040 | TGTCTTCATGCTGGTGCAGC | SEQ ID NO: 42 |
| 1023 | 1042 | TATGTCTTCATGCTGGTGCA | SEQ ID NO: 43 |
| 1025 | 1044 | GGTATGTCTTCATGCTGGTG | SEQ ID NO: 44 |
| 1032 | 1051 | TTAGCTCGGTATGTCTTCAT | SEQ ID NO: 45 |
| 1034 | 1053 | ATTTAGCTCGGTATGTCTTC | SEQ ID NO: 46 |
| 1036 | 1055 | GAATTTAGCTCGGTATGTCT | SEQ ID NO: 47 |
| 1043 | 1062 | CTCCACAGAATTTAGCTCGG | SEQ ID NO: 48 |
| 1057 | 1076 | GCCGTCGGTACATACTCCAC | SEQ ID NO: 49 |
| 1061 | 1080 | ATCGGCCGTCGGTACATACT | SEQ ID NO: 50 |
| 1064 | 1083 | AGCATCGGCCGTCGGTACAT | SEQ ID NO: 51 |
| 1066 | 1085 | GCAGCATCGGCCGTCGGTAC | SEQ ID NO: 52 |
| 1068 | 1087 | GTGCAGCATCGGCCGTCGGT | SEQ ID NO: 53 |
| 1070 | 1089 | GGGTGCAGCATCGGCCGTCG | SEQ ID NO: 54 |
| 1107 | 1126 | CACTTGAACTCCACCGGCAG | SEQ ID NO: 55 |
| 1109 | 1128 | GGCACTTGAACTCCACCGGC | SEQ ID NO: 56 |
| 1111 | 1130 | AGGGCACTTGAACTCCACCG | SEQ ID NO: 57 |
| 1113 | 1132 | TCAGGGCACTTGAACTCCAC | SEQ ID NO: 58 |
| 1116 | 1135 | CCGTCAGGGCACTTGAACTC | SEQ ID NO: 59 |
| 1118 | 1137 | CGCCGTCAGGGCACTTGAAC | SEQ ID NO: 60 |
| 1120 | 1139 | CTCGCCGTCAGGGCACTTGA | SEQ ID NO: 61 |
| 1122 | 1141 | ACCTCGCCGTCAGGGCACTT | SEQ ID NO: 62 |
| 1124 | 1143 | TGACCTCGCCGTCAGGGCAC | SEQ ID NO: 63 |
| 1126 | 1145 | CATGACCTCGCCGTCAGGGC | SEQ ID NO: 64 |
| 1132 | 1151 | CTTCTTCATGACCTCGCCGT | SEQ ID NO: 65 |
| 1134 | 1153 | TTCTTCTTCATGACCTCGCC | SEQ ID NO: 66 |
| 1159 | 1178 | GGCACAGGTCTTGATGAACA | SEQ ID NO: 67 |
| 1164 | 1183 | TGGCAGGCACAGGTCTTGAT | SEQ ID NO: 68 |
| 1166 | 1185 | AATGGCAGGCACAGGTCTTG | SEQ ID NO: 69 |
| 1168 | 1187 | GTAATGGCAGGCACAGGTCT | SEQ ID NO: 70 |
| 1170 | 1189 | TTGTAATGGCAGGCACAGGT | SEQ ID NO: 71 |
| 1172 | 1191 | AGTTGTAATGGCAGGCACAG | SEQ ID NO: 72 |
| 1175 | 1194 | GACAGTTGTAATGGCAGGCA | SEQ ID NO: 73 |
| 1179 | 1198 | CCGGGACAGTTGTAATGGCA | SEQ ID NO: 74 |
| 1181 | 1200 | CTCCGGGACAGTTGTAATGG | SEQ ID NO: 75 |
| 1183 | 1202 | GTCTCCGGGACAGTTGTAAT | SEQ ID NO: 76 |
| 1185 | 1204 | TTGTCTCCGGGACAGTTGTA | SEQ ID NO: 77 |
| 1187 | 1206 | CATTGTCTCCGGGACAGTTG | SEQ ID NO: 78 |
| 1189 | 1208 | GTCATTGTCTCCGGGACAGT | SEQ ID NO: 79 |
| 1191 | 1210 | ATGTCATTGTCTCCGGGACA | SEQ ID NO: 80 |
| 1193 | 1212 | AGATGTCATTGTCTCCGGGA | SEQ ID NO: 81 |
| 1195 | 1214 | AAAGATGTCATTGTCTCCGG | SEQ ID NO: 82 |
| 1197 | 1216 | TCAAAGATGTCATTGTCTCC | SEQ ID NO: 83 |
| 1203 | 1222 | AGCGATTCAAAGATGTCATT | SEQ ID NO: 84 |
| 1211 | 1230 | TGTAGTACAGCGATTCAAAG | SEQ ID NO: 85 |
| 1213 | 1232 | CCTGTAGTACAGCGATTCAA | SEQ ID NO: 86 |
| 1228 | 1247 | GTCTCCGTACATCTTCCTGT | SEQ ID NO: 87 |
| 1236 | 1255 | CATGCCATGTCTCCGTACAT | SEQ ID NO: 88 |
| 1238 | 1257 | TTCATGCCATGTCTCCGTAC | SEQ ID NO: 89 |
| 1240 | 1259 | GCTTCATGCCATGTCTCCGT | SEQ ID NO: 90 |
| 1242 | 1261 | TGGCTTCATGCCATGTCTCC | SEQ ID NO: 91 |
| 1244 | 1263 | TCTGGCTTCATGCCATGTCT | SEQ ID NO: 92 |
| 1246 | 1265 | TCTCTGGCTTCATGCCATGT | SEQ ID NO: 93 |
| 1329 | 1348 | ACTTGTGCTACTGAAATCAT | SEQ ID NO: 94 |
| 1331 | 1350 | TAACTTGTGCTACTGAAATC | SEQ ID NO: 95 |
| 1514 | 1533 | TCCCACTGCTCCTAAAGCCA | SEQ ID NO: 96 |
| 1516 | 1535 | CCTCCCACTGCTCCTAAAGC | SEQ ID NO: 97 |
| 1518 | 1537 | ACCCTCCCACTGCTCCTAAA | SEQ ID NO: 98 |
| 1520 | 1539 | GTACCCTCCCACTGCTCCTA | SEQ ID NO: 99 |
| 1522 | 1541 | TGGTACCCTCCCACTGCTCC | SEQ ID NO: 100 |
| 1524 | 1543 | GCTGGTACCCTCCCACTGCT | SEQ ID NO: 101 |
| 1527 | 1546 | TCTGCTGGTACCCTCCCACT | SEQ ID NO: 102 |
| 1529 | 1548 | TTTCTGCTGGTACCCTCCCA | SEQ ID NO: 103 |
| 1531 | 1550 | CCTTTCTGCTGGTACCCTCC | SEQ ID NO: 104 |
| 1533 | 1552 | AACCTTTCTGCTGGTACCCT | SEQ ID NO: 105 |
| 1574 | 1593 | AGCAGGCATATTACTCGTAT | SEQ ID NO: 106 |
| 1617 | 1636 | CAGTGAGCACGCTAAAATTT | SEQ ID NO: 107 |
| 1623 | 1642 | GCAGGTCAGTGAGCACGCTA | SEQ ID NO: 108 |
| 1625 | 1644 | AGGCAGGTCAGTGAGCACGC | SEQ ID NO: 109 |
| 1627 | 1646 | ACAGGCAGGTCAGTGAGCAC | SEQ ID NO: 110 |
| 1629 | 1648 | CTACAGGCAGGTCAGTGAGC | SEQ ID NO: 111 |
| 1631 | 1650 | GGCTACAGGCAGGTCAGTGA | SEQ ID NO: 112 |
| 1633 | 1652 | GGGGCTACAGGCAGGTCAGT | SEQ ID NO: 113 |
| 1635 | 1654 | CTGGGGCTACAGGCAGGTCA | SEQ ID NO: 114 |
| 1637 | 1656 | CACTGGGGCTACAGGCAGGT | SEQ ID NO: 115 |
| 1643 | 1662 | AGCTGTCACTGGGGCTACAG | SEQ ID NO: 116 |
| 1651 | 1670 | CACATCCTAGCTGTCACTGG | SEQ ID NO: 117 |
| 1653 | 1672 | TGCACATCCTAGCTGTCACT | SEQ ID NO: 118 |
| 1750 | 1769 | GATTCCTGAACAGTGTCATT | SEQ ID NO: 119 |
| 1752 | 1771 | CCGATTCCTGAACAGTGTCA | SEQ ID NO: 120 |
| 1755 | 1774 | ATTCCGATTCCTGAACAGTG | SEQ ID NO: 121 |
| 1759 | 1778 | CAGGATTCCGATTCCTGAAC | SEQ ID NO: 122 |
| 1761 | 1780 | GACAGGATTCCGATTCCTGA | SEQ ID NO: 123 |
| 1763 | 1782 | TCGACAGGATTCCGATTCCT | SEQ ID NO: 124 |
| 1765 | 1784 | AATCGACAGGATTCCGATTC | SEQ ID NO: 125 |
| 1767 | 1786 | CTAATCGACAGGATTCCGAT | SEQ ID NO: 126 |
| 1771 | 1790 | CAGTCTAATCGACAGGATTC | SEQ ID NO: 127 |
| 1773 | 1792 | TCCAGTCTAATCGACAGGAT | SEQ ID NO: 128 |
| 1775 | 1794 | TGTCCAGTCTAATCGACAGG | SEQ ID NO: 129 |
| 1777 | 1796 | GCTGTCCAGTCTAATCGACA | SEQ ID NO: 130 |
| 1779 | 1798 | AAGCTGTCCAGTCTAATCGA | SEQ ID NO: 131 |
| 1781 | 1800 | ACAAGCTGTCCAGTCTAATC | SEQ ID NO: 132 |
| 1783 | 1802 | CCACAAGCTGTCCAGTCTAA | SEQ ID NO: 133 |
| 1785 | 1804 | TGCCACAAGCTGTCCAGTCT | SEQ ID NO: 134 |
| 1787 | 1806 | CTTGCCACAAGCTGTCCAGT | SEQ ID NO: 135 |
| 1789 | 1808 | CACTTGCCACAAGCTGTCCA | SEQ ID NO: 136 |
| 1793 | 1812 | AATTCACTTGCCACAAGCTG | SEQ ID NO: 137 |
| 1795 | 1814 | CAAATTCACTTGCCACAAGC | SEQ ID NO: 138 |
| 1797 | 1816 | GGCAAATTCACTTGCCACAA | SEQ ID NO: 139 |
| 1799 | 1818 | CAGGCAAATTCACTTGCCAC | SEQ ID NO: 140 |
| 1801 | 1820 | TACAGGCAAATTCACTTGCC | SEQ ID NO: 141 |
| 2091 | 2110 | CACTGATGCCTCCCCTTTGC | SEQ ID NO: 142 |
| 2215 | 2234 | AACAAATGCTTCCAGGTGAA | SEQ ID NO: 143 |

Other preferred antisense oligonucleotides for use in the methods of the invention are disclosed in PCT/US02/38618; PCT/US2009/054973; PCT/US2009/054974; PCT/US2009/054975; PCT/US2009/054976; PCT/US2012/023620; U.S. Pat. No. 6,965,025 and U.S. patent application Ser. No. 13/364,547, incorporated herein by reference in their entirety.

In some embodiments, the oligonucleotides used to decrease the expression of human CTGF mRNA are small interfering RNA (siRNA). As used herein, the terms “small interfering RNA” or “siRNA” refer to single- or double-stranded RNA molecules that induce the RNA interference (RNAi) pathway and act in concert with host proteins, e.g., RNA induced silencing complex (RISC) to degrade mRNA in a sequence-specific fashion. In naturally occurring RNAi, a double-stranded RNA (dsRNA) is cleaved by the RNase III/helicase protein, Dicer, into small interfering RNA (siRNA) molecules. These siRNAs are incorporated into a multicomponent-ribonuclease called RNA-induced silencing complex (RISC). One strand of siRNA remains associated with RISC and guides the complex toward a cognate RNA that has sequence complementary to the guider ss-siRNA in RISC. This siRNA-directed endonuclease digests the RNA, thereby inactivating it.

Selective silencing of CTGF expression by RNAi can be achieved by administering isolated siRNA oligonucleotides or by the in vivo expression of engineered RNA precursors (see U.S. Pat. Nos. 7,056,704, 7,078,196, 7,459,547, 7,691,995 and 7,691,997).

In some embodiments, methods are provided to treat brain tumors wherein patients are administered siRNAs that can be incorporated into RISC structures and degrade CTGF mRNA. In some embodiments, the siRNAs are single stranded. In further embodiments, the single stranded siRNAs are at least 12, 13, 14, 15, 16, 17, 18, 19, 20, 21, 22, 23, 24, 25, 26, 27, 28, 29 or 30 nucleotides in length. In some embodiments, the siRNAs are double stranded. In further embodiments, the shortest strand of the double stranded siRNA is at least 8, 9, 10, 11, 12, 13, 14, 15, 16, 17, 18, 19, 20, 21, 22, 23, 24, 25, 26, 27, 28, 29 or 30 nucleotides in length. In other embodiments, the double stranded siRNA comprises of a guide strand, with a minimal length of 14 nucleotides. In additional embodiments, the double stranded siRNA comprises a passenger strand of at least 6, 7, 8, 9, 10, 11, 12, 13, 14, 15, 16, 17 or 18 nucleotides in length. In still further embodiments, the double stranded siRNA has a double stranded region and a single stranded region, wherein the single stranded region is 4-12 nucleotides in length. In other embodiments, the single stranded region of the siRNA has at least 1, 2, 3, 4, 5, 6, 7, 8, 9, 10, 11 or 12 nucleotide backbone modifications. In some embodiments, the double stranded siRNA comprises a guide strand and a passenger strand, wherein the double stranded region of the molecule is from 8-15 nucleotides long. In other embodiments, the double stranded siRNA has at least one blunt end or includes at least one, one or two nucleotide overhang.

In some embodiments, the siRNA is a double stranded siRNA and the sense strand of the double stranded siRNA comprises at least 12 contiguous nucleotides selected from the sequences shown in Table 2.

TABLE 2
CTGF siRNA Sense Strand Sequences

| SEQ ID NO | Start Site | Sense Sequence |
|---|---|---|
| SEQ ID NO: 144 | 1442 | UGAAGAAUGUUAA |
| SEQ ID NO: 145 | 1557 | AGAUAGCAUCUUAA |
| SEQ ID NO: 146 | 1770 | CUGUCGAUUAGAA |
| SEQ ID NO: 147 | 1815 | AACAAGCCAGAUUA |
| SEQ ID NO: 148 | 1984 | AGGUAGAAUGUAA |
| SEQ ID NO: 149 | 2265 | UGUUGAGAGUGUA |
| SEQ ID NO: 150 | 2268 | UGAGAGUGUGAA |
| SEQ ID NO: 151 | 2272 | AGUGUGACCAAAA |
| SEQ ID NO: 152 | 2273 | GUGUGACCAAAAA |
| SEQ ID NO: 153 | 2274 | UGUGACCAAAAGA |
| SEQ ID NO: 154 | 2275 | GUGACCAAAAGUA |
| SEQ ID NO: 155 | 2277 | GACCAAAAGUUAA |
| SEQ ID NO: 156 | 2295 | UGCACCUUUCUAA |
| SEQ ID NO: 157 | 2296 | GCACCUUUCUAGA |
| SEQ ID NO: 158 | 2299 | CCUUUCUAGUUGA |

In a further embodiment, the sense strand of a double stranded siRNA comprises at least 12 contiguous nucleotides of the sequence GCACCUUUCUAGA (SEQ ID NO: 159). In another embodiment, the antisense strand of a double stranded siRNA comprises at least 12 contiguous nucleotides of the sequence UCUAGAAAGGUGCAAACAU (SEQ ID NO: 160). In a further embodiment, the sense strand of a double stranded siRNA comprises at least 12 contiguous nucleotides of the sequence UUGCACCUUUCUAA (SEQ ID NO: 161). In an additional embodiment, the antisense strand of a double stranded siRNA comprises at least 12 contiguous nucleotides of the sequence UUAGAAAGGUGCAAACAAGG (SEQ ID NO: 162). In a further embodiment, the target sequence for siRNAs useful in the disclosed methods comprises at least 12 nucleotides from a target sequence shown in Table 3.

TABLE 3
CTGF Target Sequences for siRNA

| | Start | |
|---|---|---|
| SEQ ID NO | Site | Target Sequence |
| SEQ ID NO: 163 | 379 | GGGCCUCUUCUGUGACUUC |
| SEQ ID NO: 164 | 691 | CCGACUGGAAGACACGUUU |
| SEQ ID NO; 165 | 801 | CCCGGGUUACCAAUGACAA |
| SEQ ID NO: 166 | 901 | GGGCAAAAAGUGCAUCCGU |
| SEQ ID NO: 167 | 932 | UCCAAGCCUAUCAAGUUUGAGCUUU |
| SEQ ID NO: 168 | 937 | GCCUAUCAAGUUUGAGCUU |
| SEQ ID NO: 169 | 969 | GCAUGAAGACAUACCGAGCUAAAUU |
| SEQ ID NO: 170 | 986 | GCUAAAUUCUGUGGAGUAU |
| SEQ ID NO: 171 | 1119 | GCCAUUACAACUGUCCCGGAGACAA |
| SEQ ID NO: 172 | 1170 | GGAAGAUGUACGGAGACAU |
| SEQ ID NO: 173 | 1201 | GAGAGUGAGAGACAUUAACUCAUUA |
| SEQ ID NO: 174 | 1346 | GCCAUGUCAAACAAAUAGUCUAUCU |
| SEQ ID NO: 175 | 1473 | GGGUACCAGCAGAAAGGUU |
| SEQ ID NO: 176 | 1478 | CCAGCAGAAAGGUUAGUAU |
| SEQ ID NO: 177 | 1481 | GCAGAAAGGUUAGUAUCAU |
| SEQ ID NO: 178 | 1488 | GGUUAGUAUCAUCAGAUAG |
| SEQ ID NO: 179 | 1626 | GAGACUGAGUCAAGUUGUUCCUUAA |
| SEQ ID NO: 180 | 1660 | GCAGACUCAGCUCUGACAU |
| SEQ ID NO: 181 | 1666 | UCAGCUCUGACAUUCUGAUUCGAAU |
| SEQ ID NO: 182 | 1712 | UCCUGUCGAUUAGACUGGACAGCUU |
| SEQ ID NO: 183 | 1733 | GCUUGUGGCAAGUGAAUUU |

Cellular uptake of siRNA can be improved by increasing the hydrophobicity of compounds, i.e., through the use of hydrophobic base modifications. (See PCT/US2011/029824, PCT/US2011/029849 and PCT/US2011/029867).

In some embodiments, treatment methods are provided wherein patients are administered a recombinant expression vector that expresses anti-CTGF antisense or anti-CTGF siRNA precursors. Such genetic constructs can be designed using appropriate vectors and expressional regulators for cell- or tissue-specific expression and constitutive or inducible expression. These genetic constructs can be formulated and administered according to established procedures within the art. In some embodiments, patients are administered recombinant expression vectors that encode a short hairpin oligonucleotide. In further embodiments, the recombinant expression vectors are DNA plasmids, while in other embodiments, the expression vectors are viral vectors. RNA expressing viral vectors can be constructed based on, but not limited to, adeno-associated viruses, retroviruses, adenoviruses, or alphaviruses. In some embodiments, the expression vectors persist in target cells. Alternatively, such vectors can be repeatedly administered as necessary.

### Pharmaceutical Compositions

The anti-CTGF agents of the present invention can be delivered directly or in pharmaceutical compositions containing carriers and excipients, as is well known in the art. Pharmaceutical compositions of the invention include compositions suitable for injectable use and compositions suitable for incorporation into semi-solid or solid dosage forms.

The compositions can be liquid solutions, suspensions, emulsions, tablets, pills, capsules, sustained release formulations, or powders. Injectable forms include sterile aqueous solutions, dispersions and sterile powders for the extemporaneous preparation of sterile injectable solutions or dispersions. Various formulations and drug delivery systems are available in the art. (See, e.g., Gennaro, ed. (2000) Remington's Pharmaceutical Sciences, supra, and Goodman and Gilman's The Pharmacological Basis of Therapeutics, 10^{th} Ed. (2001), Hardman, Limbird, and Gilman, eds. MacGraw Hill Intl.)

For the purpose of the invention, suitable pharmaceutically acceptable carriers include, but are not limited to: water, salt solutions (e.g., NaCI), alcohols, gum arabic, vegetable oils, benzyl alcohols, polyethylene glycols, gelatin, carbohydrates such as lactose, amylose or starch, magnesium stearate, talc, silicic acid, viscous paraffin, fatty acid esters, hydroxymethylcellulose, and polyvinyl pyrolidone. The pharmaceutical compositions can be, if desired, mixed with excipients, e.g., lubricants, preservatives, stabilizers, wetting agents, emulsifiers, salts for influencing osmotic pressure, buffers, coloring and the like which do not deleteriously react with the active compounds. In some embodiments, the pharmaceutical compositions of the invention are liquid compositions that can be administered by intravenous, intrathecal, intracerebral or intratumoral injection.

In further embodiments, the anti-CTGF agent is associated with a carrier or vehicle, e.g., liposomes or micelles, although other carriers could be used, as would be appreciated by one skilled in the art. Liposomes are vesicles made of a lipid bilayer having a structure similar to biological membranes. Such carriers are used to facilitate the cellular uptake or targeting of the anti-CTGF agent, or improve the anti-CTGF agent's pharmacokinetic or toxicologic properties. For example, the anti-CTGF oligonucleotides useful in the methods of the present invention may be administered encapsulated in liposomes.

In particular embodiments, the present invention provides pharmaceutical compositions for therapeutically treating a brain tumor comprising an anti-CTGF agent or a biologically active derivative thereof including anti-CTGF antibody fragments or anti-CTGF antibody mimetics, i.e., small binding molecules that are designed to mimic an antibody or antibody fragment such as AdNectins, Affibodies, Anticalins or Kunitzs. In a particular embodiment of the invention, the isolated anti-CTGF antibody incorporated into the pharmaceutical composition of the invention is identical to CLN-1 or a biologically active fragment thereof. In further embodiments, the pharmaceutical compositions comprise an isolated anti-CTGF oligonucleotide and a pharmaceutically acceptable carrier.

In some embodiments, the anti-CTGF agent is formulated as or loaded onto a semi-solid or solid substrate. In further embodiments, the semi-solid or solid substrate is biodegradable in the brain. In other embodiments, the semi-solid or solid substrate releases the anti-CTGF agent in a controlled manner. In some embodiments, the semi-solid or solid substrates are microparticles such as microspheres than can be injected or otherwise deposited intratumorally or into a surgical cavity created by the resection of the tumor. Suitable microspheres contemplated for use in the methods of the invention are disclosed in U.S. patent application Ser. No. 12/089,797. In other embodiments, the semi-solid substrate is a soft, elastomeric, compressible hollow foam. Such foam may be shaped as spheres or other suitable shapes that following compression are placed into the resection cavity where they expand to conform to the cavity's shape. In this way maximum contact with the surface area of the tissue forming the walls of the cavity is obtained allowing all or substantially all tissue faces that comprise the cavity surface to receive high concentrations of anti-CTGF agent as it diffuses out of the foam. Exemplary hollow foam beads suitable for practicing the present invention are found in U.S. patent application Ser. No. 12/426,405.

In further embodiments, a hydrogel (also known as an aquagel) formulated to contain a therapeutically effective concentration of an anti-CTGF agent is placed into the surgical cavity. In some embodiments, the hydrogel additionally contains a polymeric drug delivery microparticles that allow for the controlled release of the anti-CTGF agent. An exemplary hydrogel for the controlled release of a drug for use in the methods of the invention is disclosed in Yeo Y et al. Ann Surg. 2007; 245: 819-824.

### Administration and Dosage

A therapeutically effective amount of an anti-CTGF agent or pharmaceutical composition thereof can be administered intravenously, intrathecally, intracerebrally, intratumorally or by intracavitary installation. Intratumoral injection can be performed, for example, by using stereotactic neurosurgery. In some embodiments, the anti-CTGF agent is administered as a bolus injection. In other embodiments, the anti-CTGF agent is administered continuously as an infusion. In particular embodiments, the anti-CTGF agent is infused continuously from a catheter or a plurality thereof into a tumor, surgical cavity or tissue surrounding a tumor using an infusion pump. In further embodiments, the pressure gradient of the infusion pump is static, while in other embodiments, the pressure gradient increases over at least part or all of the infusion period.

A therapeutically effective amount of an anti-CTGF agent or pharmaceutical composition thereof can be administered as often as necessary, e.g., once, twice or three times per day, every other day, once, twice or three times per week, every other week, every three weeks or monthly. The skilled artisan will appreciate that certain factors may influence the dosage and timing required to effectively treat a subject, including but not limited to the severity or extent of the disease, the administration route, previous treatments, concurrent medications, performance status, weight, gender, race or ethnicity, and/or age of the subject. In certain embodiments, the methods for treating brain tumors presented herein comprise the administration to a subject in need thereof an anti-CTGF agent at a range from about 0.01 mg to about 10,000 mg, from about 0.1 mg to about 5,000 mg, from about 1.0 mg to about 2,500 mg, from about 1.0 mg to about 1,000 mg, from about 10 mg to about 500 mg, from about 100 mg to about 1,000 mg, from about 0.10 mg to about 50 mg or from about 0.5 mg to about 50 mg.

In some embodiments, the methods for treating brain tumors presented herein comprise the administration to a subject in need thereof at least about 0.1 mg, 0.5 mg, 1.0 mg, 2.0 mg, 4 mg, 8 mg, 16 mg, 25 mg, 50 mg, 100 mg, 200 mg, 400 mg, 800 mg, 1,000 mg, 2,000 mg, 3,000 mg, 5,000 mg or 10,000 mg of an anti-CTGF agent. In some embodiments, the methods for treating brain tumors presented herein comprise the administration to a subject in need thereof not more than about 1 mg, 5 mg, 10 mg, 20 mg, 30 mg, 40 mg, 50 mg, 60 mg, 70 mg, 80 mg, 90 mg, 100 mg, 150 mg, 175 mg, 200 mg, 250 mg, 500 mg, 750 mg, 1,000, 2,000, 5,000 mg or 10,000 mg of an anti-CTGF agent.

In some embodiments, the methods for treating brain tumors presented herein comprise the administration to a subject in need thereof an anti-CTGF agent from about 0.001 mg/kg to about 5,000 mg/kg, about 0.01 mg/kg to about 1000 mg per kg, about 0.1 mg/kg to about 500 mg per kg or about 1.0 mg/kg to about 100 mg/kg.

In some embodiments, the methods for treating brain tumors presented herein comprise the administration of a dose of an anti-CTGF agent that is expressed as mg/m². The mg/m² for an anti-CTGF agent may be determined, for example, by multiplying a conversion factor for an animal by an animal dose in mg/kg to obtain the dose in mg/m² for human dose equivalent. Conversion factors to translate animal data to humans are known in the art and include: Mouse=3, Hamster=4.1, Rat=6, Guinea Pig=7.7. (Adapted from Freireich et al, Cancer Chemother. Rep. 50:219-244 (1966)). The height and weight of a human may be used to calculate a human body surface area applying Boyd's Formula of body surface area. In specific embodiments, the methods for treating brain tumors presented herein comprise the administration to a subject in need thereof of an amount of an anti-CTGF agent in the range of from about 0.01 mg/m² to about 10,000 mg/m², about 0.10 mg/m² to about 1,000 mg/m², or about 1.0 mg/m² to about 100 mg/m².

In some embodiments, an effective amount of an anti-CTGF antibody is administered systemically, e.g., i.v. administration. In further embodiments, the systemically administered dose comprises a dose of anti-CTGF antibody between about 1 mg/kg to 100 mg/kg, 5 mg/kg to 75 mg/kg, 10 mg/kg to 50 mg/kg, 15 mg/kg to 45 mg/kg or 20 mg/kg to 45 mg/kg. In other embodiments, an effective amount of a systemically administered anti-CTGF antibody comprises a dose of about 15 mg/kg, 20 mg/kg, 25 mg/kg, 30 mg/kg, 35 mg/kg, 40 mg/kg, 45 mg/kg or 50 mg/kg.

In some embodiments, an effective amount of an anti-CTGF antibody is administered locally, e.g., intratumoral or into the surgical cavity. In further embodiments, the locally administered dose comprises a dose between about 0.01 mg to about 1,000 mg, about 0.1 mg to about 100 mg, about 0.1 mg to about 5 mg, about 1.0 mg to about 50 mg, about 1.0 mg to about 10 mg, or 5 about mg to about 25 mg.

In some embodiments, an effective amount of an anti-CTGF oligonucleotide is administered locally, e.g., intratumoral or into the surgical cavity. In further embodiments, the locally administered dose of an anti-CTGF oligonucleotide comprises a dose between about 0.01 mg to about 1,000 mg, about 0.1 mg to about 100 mg, about 1.0 mg to about 50 mg, about 1.0 mg to about 25 mg, or about 5 mg to about 50 mg.

In some embodiments, the methods for treating brain tumors presented herein comprise the administration to a subject in need thereof of an anti-CTGF agent or a pharmaceutical composition thereof at a dosage that achieves a target plasma, cerebrospinal fluid, brain tissue, or tumor concentration of the anti-CTGF agent. In particular embodiments, the administered dosage achieves a plasma, cerebrospinal fluid, brain tissue, or tumor concentration of the anti-CTGF agent ranging from about 0.001 g/mL to about 100 mg/mL, about 0.01 μg/mL to about 10 mg/mL, about 0.1 μg/mL to about 1 mg/mL or about 1 μg/mL to about 100 μg/ml in a subject with a brain tumor.

In particular embodiments, the administration to a subject in need thereof of an anti-CTGF antibody achieves a plasma, cerebrospinal fluid, brain tissue, or tumor target concentration of the anti-CTGF antibody of at least about 10 μg/ml, about 50 μg/ml, about 100 μg/mL, about 200 μg/mL, about 300 μg/mL, or about 400 μg/mL. In further embodiments, the administration to a subject in need thereof of an anti-CTGF antibody achieves a plasma, cerebrospinal fluid, brain tissue, or tumor target concentration of a range of about 1.0 μg/ml to about 2,000 μg/ml, about 10 μg/mL to about 1,000 μg/mL, or about 20 μg/mL to about 500 μg/mL.

In certain embodiments, subsequent doses of an anti-CTGF agent may be adjusted accordingly based on the plasma, cerebrospinal fluid, brain tissue, or tumor concentration of the anti-CTGF agent achieved with earlier doses of the anti-CTGF agent. In general, the dosage and frequency of administration of an anti-CTGF agent may be adjusted over time to provide sufficient levels of the anti-CTGF agent to maintain the desired effect.

In other embodiments, the methods of the invention the anti-CTGF agent or pharmaceutical composition thereof is administered as a neoadjuvant. As used herein, the term “neoadjuvant” describes a therapeutic agent that is administered prior to a main therapy, usually surgery, typically to inhibit the growth of a tumor, or shrink the size of a tumor. Frequently, a neoadjuvant is administered to improve the outcome of surgery, by for example, making the tumor easier to excise. In some embodiments, the administration of an anti-CTGF agent prevents or decrease the migration and invasion of glioma cells into the surrounding normal tissue. In further embodiments, neoadjuvant administration results in cleaner, more distinct tumor margins. In other embodiments, neoadjuvant administration reduces tumor desmoplasia. In additional embodiments, neoadjuvant administration of an anti-CTGF agent allows a surgeon to remove less normal tissue (smaller surgical margin) compared to the amount of normal tissue typically excised using standard of care therapy. In some embodiments, administration of an anti-CTGF agent allows the tumor surgical margin to be less than 0.5 cm in width, less than 1 cm in width, less than 1.5 cm in width or less than 2.0 cm in width. In further embodiments, no more than 0.5 cm, 1.0 cm, 1.5 cm or 2.0 cm of normal white matter bordering the tumor margin is required to be excised with the tumor to produce an equivalent therapeutic outcome compared to standard therapy. A decrease in the extent of normal white matter surgical excised with a brain tumor can decrease the number or severity of neurologic deficits associated with brain tumor surgery. In a preferred embodiment, the anti-CTGF agent is administered to the patient as close to the time of diagnosis as is practical. In other embodiments, the neoadjuvant is administered about 1 day, 2 days, 3 days, 4 days, 5 days, 6 days, 7 days, 8 days, 9 days, 10 days, 11 days, 12 days, or 14 days before surgery. In further embodiments, the neoadjuvant is administered about 1 day to 7 days, 1 day to 14 days, or 7 days to 14 days before surgery.

In some embodiments, the anti-CTGF agent or pharmaceutical composition is administered during or after surgery to prevent or inhibit the growth of tumor cells that were shed during the procedure, tumor masses that were not substantially resectable and/or tumor cells that had invaded adjacent normal tissue that was not excised. Tumor masses that are not substantially resectable include tumor masses affixed to or infiltrated into critical nervous system structures. Tumor masses that are not substantially resectable include tumor masses wherein at least about 5%, at least 10%, at least 15%, at least 20%, at least 25%, at least 30%, at least 35%, at least 40% or at least about 50% or more of the estimated tumor volume cannot be safely excised. Tumor masses that are not substantially resectable further include tumors where about 1.0 cm³ or more residual tumor remains after surgery. Invasive tumor cells can exist as single cells, or as discrete tumor cell masses or clusters. In some embodiments, treatment with an anti-CTGF agent prevents or decreases the growth of shed tumor cells, non-excisable tumor masses or invasive tumor cells. In further embodiments, treatment with an anti-CTGF agent decreases the growth rate of shed tumor cells, non-excisable tumor masses or invasive tumor cells by at least 10%, 20%, 30%, 40%, 50%, 60%, 70%, 80%, 90% or 95% compared to a control group or historical controls. In some embodiments, residual tumor growth is monitored by radiographic criteria.

In some embodiments, the methods of the invention comprising the administration of an effective amount of an anti-CTGF agent prolongs the survival of treated brain tumor patients. In further embodiments, the methods comprising the administration of an effective amount of an anti-CTGF agent prolongs the survival of brain tumor patients by at least 10%, 20%, 30%, 40%, 50%, 60%, 70%, 80%, 90% or 100% compared to a control group or historical controls. In other embodiments, the methods comprising the administration of an anti-CTGF agent prolongs survival of brain tumor patients by at least two weeks, at least 1 month, at least 2 months, at least 3 months, at least 4 months, at least 5 months, at least 6 months, at least 7 months, at least 8 months, at least 9 months, at least 10 months, at least 11 months, at least 12 months, at least 15 months, at least 18 months, at least 24 months at least 3 years, at least 4 years or at least 5 years compared to a control group or historical control. Patient survival can be measured in terms of disease-free survival, progression-free survival or overall survival. In some embodiments, the prolongation of survival produces a progression-free survival rate for GBM patients of at least 8 months, 9 months, 10 months, 11 months, 12 months, 13 months, 14 months, 15 months, 16 months or 18 months. In further embodiments, the prolongation of survival produces an overall survival rate for GBM patients of at least 16 months, 18 months, 20 months, 22 months, 24 months, 30 months or 36 months. In other embodiments, the one-year survival of GBM patients treated with the methods of the invention comprising the administration of an anti-CTGF agent is at least 55%, 60%, 65%, 70%, 75%, 80%, 85%, 90% or 95%. In further embodiments, the prolongation of survival results in a two year survival rate for subjects with GBM of at least 15%, 20%, 25%, 30%, 35%, 40%, 50%, 60% or 70%. In some embodiments, newly diagnosed GBM patients treated with the methods of the invention comprising the administration of an anti-CTGF agent have a median survival of at least 13 months, 14 months, 15 months, 16 months, 17 months, 18 months, 19 months, 20 months, 21 months, 22, months, 23 months, 24 months, 26 months, 28 months or 30 months. In further embodiments, the two-year survival rate for anaplastic astrocytoma patients treated with the methods of the invention comprising the administration of an anti-CTGF agent is at least 55%, 60%, 65%, 70%, 75%, 80%, 85%, 90% or 95%. In additional embodiments, the prolongation of survival results in a three year survival rate of at least 15%, 20%, 25%, 30%, 35%, 40%, 50%, 60% or 70% for subjects with anaplastic astrocytoma. In some embodiments, treatment with the methods of the invention comprising the administration of an anti-CTGF agent delays the recurrence of the brain tumor by least four weeks, at least six weeks, at least eight weeks, at least ten weeks, at least twelve weeks, at least eighteen weeks, at least twenty four weeks, at least thirty weeks, at least forty eight weeks, at least fifty two weeks, at least 2 years, 3 years, 4 years or 5 years

### Combination Therapy

In some embodiments, the methods of the invention for treating brain tumors provided herein comprise administering an anti-CTGF agent with one or more additional therapies. Current therapies for brain tumors include surgery, radiation and/or chemotherapy. Therefore, in specific embodiments, the methods of the invention comprise the administration of an anti-CTGF agent and radiation therapy, including carbon ion, proton or photon radiotherapy, gamma knife surgery or brachytherapy. In other embodiments, the methods of the invention comprise the administration of an anti-CTGF agent and surgery to excise part, most, substantially all, or all of a brain tumor. In further embodiments, the methods of the invention comprise the administration of an anti-CTGF agent and chemotherapy including implantable chemotherapy depots. In still further embodiments, the methods of the invention comprise the administration of an anti-CTGF agent and two or more of the following: surgery, radiation therapy, chemotherapy, immunotherapy or other biologic agents.

In some embodiments, the methods of the invention comprising the administration of an anti-CTGF agent further comprise the administration of a hormonal agent, chemotherapy agent (e.g., microtubule dissembly blocker, antimetabolite, topisomerase inhibitor, and DNA crosslinker or damaging agent), anti-angiogenic agent (e.g., VEGF antagonist), or immunotherapy agent, for example, dendritic cell therapy or cancer vaccines.

Non-limiting examples of hormonal agents that may be used with an anti-CTGF agent include aromatase inhibitors, SERMs, and estrogen receptor antagonists. Hormonal agents that are aromatase inhibitors may be steroidal or nonsteroidal. Non-limiting examples of nonsteroidal hormonal agents include letrozole, anastrozole, aminoglutethimide, fadrozole, and vorozole. Non-limiting examples of steroidal hormonal agents include aromasin (exemestane), formestane, and testolactone. Non-limiting examples of hormonal agents that are SERMs include tamoxifen, afimoxifene, arzoxifene, bazedoxifene, clomifene, femarelle, lasofoxifene, ormeloxifene, raloxifene, and toremifene. Non-limiting examples of hormonal agents that are estrogen receptor antagonists include fulvestrant. Other hormonal agents include but are not limited to abiraterone and lonaprisan.

Non-limiting examples of chemotherapy agents that may be used with an anti-CTGF agent include microtubule disassembly blocker, antimetabolite, topisomerase inhibitor, and DNA crosslinker or damaging agent. Chemotherapy agents that are microtubule dissemby blockers include, but are not limited to, taxenes (e.g., paclitaxel), docetaxel, abraxane, larotaxel, ortataxel, and tesetaxel); epothilones (e.g., ixabepilone); and vinca alkaloids (e.g., vinorelbine, vinblastine, vindesine, and vincristine (branded/marketed as ONCOVIN®)).

Chemotherapy agents that are antimetabolites include, but are not limited to, folate antimetabolites (e.g., methotrexate, aminopterin, pemetrexed, raltitrexed); purine antimetabolites (e.g., cladribine, clofarabine, fludarabine, mercaptopurine, pentostatin, thioguanine); pyrimidine antimetabolites (e.g., 5-fluorouracil, capcitabine, gemcitabine, cytarabine, decitabine, floxuridine, tegafur); and deoxyribonucleotide antimetabolites (e.g., hydroxyurea).

Chemotherapy agents that are topoisomerase inhibitors include, but are not limited to, class I (camptotheca) topoisomerase inhibitors (e.g., topotecan, irinotecan, rubitecan, and belotecan); class II (podophyllum) topoisomerase inhibitors (e.g., etoposide or VP-16, and teniposide); anthracyc lines (e.g., doxorubicin, epirubicin, Doxil, aclarubicin, amrubicin, daunorubicin, idarubicin, pirarubicin, valrubicin, and zorubicin); banoxantrone (AQ4N), RTA 744, and anthracenediones (e.g., mitoxantrone, and pixantrone).

Chemotherapy agents that are DNA crosslinkers (or DNA damaging agents) include, but are not limited to, alkylating agents (e.g., cyclophosphamide, mechlorethamine, ifosfamide, trofosfamide, chlorambucil, melphalan, prednimustine, bendamustine, uramustine, estramustine, carmustine, lomustine, semustine, fotemustine, nimustine, ranimustine, streptozocin, busulfan, mannosulfan, treosulfan, carboquone, N,N′N′-triethylenethiophosphoramide, triaziquone, triethylenemelamine); alkylating-like agents (e.g., carboplatin (branded/marketed as PARAPLATIN®), cisplatin, oxaliplatin, nedaplatin, triplatin tetranitrate, satraplatin, picoplatin); nonclassical DNA crosslinkers (e.g., procarbazine, dacarbazine, temozolomide (branded/marketed as TEMODAR®), altretamine, mitobronitol); and intercalating agents (e.g., actinomycin, bleomycin, mitomycin, and plicamycin).

In some embodiments of the invention, the methods comprising the administration of an anti-CTGF agent further comprises the administration of a chemotherapy agent selected from the group consisting of Acivicin; Aclarubicin; Acodazole Hydrochloride; Acronine; Adriamycin; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azathioprine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Capecitabine; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxifluridine; Doxorubicin; Doxorubicin Hydrochloride; Droloxifene; Droloxifene Citrate; Dromostanolone Propionate; Duazomycin; Edatrexate; Eflonithine Hydrochloride; Elsamitrucin; Enloplatin; Enpromate; Epipropidine; Epirubicin Hydrochloride; Epothilone; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Fluorocitabine; Fosquidone; Fostriecin Sodium; Gemcitabine; Gemcitabine Hydrochloride; Hydroxyurea; Idarubicin Hydrochloride; Ifosfamide; Ilmofosine; Imatinib; Interferon Alfa-2a; Interferon Alfa-2b; Interferon Alfa-n1; Interferon Alfa-n3; Interferon Beta-Ia; Interferon Gamma-Ib; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper, Mitotane; Mitoxantrone Hydrochloride; Mycophenolic Acid; Nocodazole; Nogalamycin; Ormaplatin; Oxaliplatin; Oxisuran; Paclitaxel; Pegaspargase; Peliomycin; Pemetrexed; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; all-trans retinoic acid; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Taxol; Tecogalan Sodium; Tegafur; Teloxantrone Hydrochloride; Temoporfin; Teniposide; Teroxirone; Testolactone; Thiamiprine; Thioguanine; Thiotepa; Tiazofuirin; Tioguanine; Tirapazamine; Topotecan Hydrochloride; Toremifene Citrate; Trestolone Acetate; Triciribine Phosphate; Trimetrexate; Temozolomide (Temodare®); Dorafinib, Sorafenib (Nexavar), Sunitinib, Vandetanib (ZD6474), Pazopanib (GW786034), Vatalanib (PTK787), Trimetrexate Glucuronate; Triptorelin; Tubulozole Hydrochloride; Uracil Mustard; Uredepa; Valrubicin; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorzole; Zeniplatin; Zinostatin; and Zorubicin Hydrochloride. Additional chemotherapy agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division).

In specific embodiments, the methods comprising the administration of an anti-CTGF agent further comprises the administration of at least one chemotherapy agent selected from the group consisting of temozolmide, procarbazine, lomustine, vincristine, cisplatin, carmustine, carboplatin and methotrexate.

In further embodiments, the methods of the invention comprising the administration with an anti-CTGF agent further comprises the administration of an anti-angiogenic agent, including, but not limited to, VEGF antagonists, VEGF receptor antagonists, integrin antagonists (e.g., vitaxin, cilengitide, and S247), and VTAs/VDAs (e.g., fosbretabulin). VEGF antagonists include, but are not limited to, anti-VEGF antibodies (e.g., bevacizumab and ranibizumab), VEGF traps (e.g., aflibercept), VEGF antisense or siRNA or miRNA, and aptamers (e.g., pegaptanib). Anti-angiogenic agents that are receptor antagonists include, but are not limited to, antibodies (e.g., ramucirumab) and kinase inhibitors (e.g., sunitinib, sorafenib, cediranib, panzopanib, vandetanib, axitinib, and AG-013958). Other non-limiting examples of anti-angiogenic agents include ATN-224, anecortave acetate, microtubule depolymerization inhibitors such as combretastatin A4 prodrug, and recombinant proteins or protein fragments such as collagen 18 (endostatin).

In additional embodiments, the methods of the invention comprising the administration of an anti-CTGF agent further comprises the administration of (1) a statin such as lovastatin, (2) an mTOR inhibitor such as sirolimus which is also known as Rapamycin, temsirolimus, evorolimus, and deforolimus; (3) a farnesyltransferase inhibitor agent such as tipifarnib; (4) an antifibrotic agent such as pirfenidone; (5) a pegylated interferon such as PEG-interferon alpha-2b; (6) a CNS stimulant such as methylphenidate; (7) a HER-2 antagonist such as anti-HER-2 antibody (e.g., trastuzumab) or kinase inhibitor (e.g., lapatinib); (8) an IGF-I antagonist such as an anti-IGF-1 antibody (e.g., AVE 1642 and IMC-A11) or an IGF-I kinase inhibitor, (9) EGFR/HER-1 antagonist such as an anti-EGFR antibody (e.g., cetuximab, panitumamab) or EGFR kinase inhibitor (e.g., erlotinib, gefitinib); (10) SRC antagonist such as bosutinib; (11) cyclin dependent kinase (CDK) inhibitor such as seliciclib; (12) Janus kinase 2 inhibitor such as lestaurtinib; (13) proteasome inhibitor such as bortezomib; (14) phosphodiesterase inhibitor such as anagrelide; (15) inosine monophosphate dehydrogenase inhibitor such as tiazofurine; (16) lipoxygenase inhibitor such as masoprocol; (17) endothelin antagonist; (18) retinoid receptor antagonist such as tretinoin or alitretinoin; (19) immune modulator such as lenalidomide, pomalidomide, or thalidomide; (20) kinase (e.g., tyrosine kinase) inhibitor such as imatinib, dasatinib, erlotinib, nilotinib, gefitinib, sorafenib, sunitinib, lapatinib, AEE788, or TG 100801; (21) non-steroidal anti-inflammatory agent such as celecoxib; (22) human granulocyte colony-stimulating factor (G-CSF) such as filgrastim; (23) folinic acid or leucovorin calcium; (24) integrin antagonist such as an integrin α5β1-antagonist (e.g., JSM6427); (25) nuclear factor kappa beta (NF-Kβ) antagonist such as OT-551; (26) hedgehog inhibitor such as CUR61414, cyclopamine, GDC-0449, or anti-hedgehog antibody; (27) histone deacetylase (HDAC) inhibitor such as SAHA, PCI-24781, SB939, CHR-3996, CRA-024781, ITF2357, JNJ-26481585, or PCI-24781; (28) retinoid such as isotretinoin; (29) hepatocyte growth factor/scatter factor (HGF/SF) antagonist such as HGF/SF monoclonal antibody (e.g., AMG 102) or a c-Met kinase inhibitor such as crizotinib; (30) synthetic bradykinin such as RMP-7; (31) platelet-derived growth factor receptor inhibitor such as SU-101; (32) receptor tyrosine kinase inhibitors of Flk-1/KDR/VEGFR2, FGFR1 and PDGFR beta such as SU5416 and SU6668; (33) anti-inflammatory agent such as sulfasalazine; (34) TGF-beta antisense or siRNA therapy, including trabedersen, an antisense oligonucleotide that is complentary to TGF-β2 mRNA; (35) oncolytic viruses, including oncolytic adenovirus d11520 (ONXY-015); or (36) a ketogenic diet.

Other therapeutic agents and modalities (e.g., surgery, radiation therapy, chemotherapy, immunotherapy or other biologic agents) can be administered prior to, concurrently with, or subsequent to the administration of an anti-CTGF agent for the treatment of brain tumors. For chemotherapy, immunotherapy and biologic agents, these additional therapies may be administered by the same route or a different route of administration than used for the anti-CTGF agent.

In specific embodiments, the interval of time between the administration of an anti-CTGF agent and the administration of one or more additional therapies may be about 1 minute to 30 minutes, 30 minutes to 60 minutes, 1 hour to 4 hours, 2 hours to 12 hours, 12 hours to 24 hours, 1 day to 2 days, 2 days to 4 days, 1 days to 7 days, 1 week to 2 weeks, 4 weeks, 6 weeks, 8 weeks, 10 weeks, 15 weeks, 20 weeks, 52 weeks or any period of time in between. In certain embodiments, an anti-CTGF agent and one or more additional therapies are administered less than 1 day, 1 week, 2 weeks, 3 weeks, 4 weeks, one month, 2 months, 3 months, 6 months or 1 year apart.

In certain embodiments, an anti-CTGF agent and one or more additional therapies are cyclically administered to a subject. In this context, cycling therapy involves the administration of the anti-CTGF agent for a period of time, followed by the administration of one or more additional therapies for a period of time. The cycles of the anti-CTGF agent and the other additional therapy can overlap or be identical in time. The cycles can be followed by an optional period of rest before repeating the cycles. The optional period of rest can be for a time period of about 1 day, 2 days, 3 days, 4 days, 5 days, 6 days, 7 days, 2 weeks, 3 weeks, 4 weeks, 5 weeks, 6 weeks, 8 weeks, or 12 weeks. In some embodiments, the number of cycles administered is from 1 to 12 cycles, from 2 to 10 cycles, or from 4 to 8 cycles.

In some embodiments, the methods of the invention comprising the administration of an anti-CTGF agent further comprises the administration of a medication for controlling or relieving symptoms associated with brain tumors, e.g., headaches, seizures, edema, proteinuria, nausea and/or vomiting. Accordingly, in some embodiments, the administration to a subject in need thereof an anti-CTGF agent further comprises the administration of at least one agent selected from the group consisting of a pain reliever, a medication for seizures, corticosteroids (e.g., dexamethasone), anticonvulsant drugs, anticoagulant drugs, anti-emetic or a 5HT3 blocker (e.g., ondansetron hydrochloride or granisetron hydrochloride), acetylcholine esterase (ACE) inhibitors (e.g., lisinopril) and angiotensin II receptor blockers (ARB).

In some embodiments, administration of an anti-CTGF agent and one or more additional therapies in accordance with the methods presented herein have an additive effect relative to the administration of the anti-CTGF agent or the one or more additional therapies alone. In other embodiments, the administration of an anti-CTGF agent and one or more additional therapies in accordance with the methods presented herein have a synergistic effect relative to the administration of the anti-CTGF agent or the one or more additional therapies alone.

As used herein, the term “synergistic,” refers to the effects achieved with the administration of an anti-CTGF agent and one or more additional therapies e.g., temozolomide or radiation, the combination being more effective than the additive effects of the CTGF agent and the one or more additional therapies. In some embodiments, a synergistic effect results in improved efficacy in treating brain tumors. In further embodiments, a synergistic effect permits the use of lower dosages (e.g., sub-optimal doses) of a cytotoxic therapy, e.g., temozolomide or radiation, and/or less frequent administration of the cytotoxic therapy while maintaining or improving upon the therapeutic effect achieved with a standard dose of the cytotoxic therapy. The decrease in dose or frequency of dosing of a cytotoxic therapy made possible by the synergistic activity an anti-CTGF agent and the cytotoxic therapy can avoid or decrease one or more adverse or unwanted side effects associated with the use of the cytotoxic therapy, e.g., decreased nausea or vomiting typically seen with temozolomide.

### Articles of Manufacture

The present compositions may, if desired, be presented in a pack or dispenser device containing one or more unit dosage forms containing an anti-CTGF agent. Such a pack or device may, for example, comprise metal or plastic foil, glass and rubber stoppers, such as in vials, or syringes. The container holds or contains an anti-CTGF agent composition that is effective for treating a brain tumor and may have a sterile access port (for example the container may be an intravenous solution bag or a vial having a stopper pierceable by a hypodermic injection needle). The article of manufacture may further comprise an additional container comprising a pharmaceutically acceptable diluent buffer, such as bacteriostatic water for injection (BWFI), phosphate-buffered saline, Ringer's solution, and/or dextrose solution. The article of manufacture may further include other materials desirable from a commercial and user standpoint, including other buffers, diluents, filters, needles, and syringes.

Compositions comprising an anti-CTGF agent formulated in a compatible pharmaceutical carrier may be provided in an appropriate container that is labeled for treatment of brain tumors. The pack or dispenser device may be accompanied by instructions for administration that provide specific guidance regarding dosing the anti-CTGF agent including a description of the type of patients who may be treated (e.g., a person with a glioma), the schedule (e.g., dose and frequency) and route of administration, and the like.

The following examples serve only to illustrate and aid in the understanding of the invention and are not intended to limit the invention as set forth in the claims which follow thereafter. Various modifications of the invention, in addition to those shown and described herein, will become apparent to those skilled in the art from the foregoing description. Such modifications are intended to fall within the scope of the appended claims. All references cited herein are hereby incorporated by reference in their entirety.

### EXAMPLES

The invention will be further understood by reference to the following examples, which are intended to be purely exemplary of the invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description and accompanying figures. Such modifications are intended to fall within the scope of the appended claims.

### Example 1

### Cell Cultures and Treatment Conditions

Human glioblastoma (U87MG and T98) tumor cells (Tumorbank DKFZ Heidelberg, Germany) were cultured in DMEM medium with 10% FCS at 37° C. with 5% CO₂ and 95% humidity. GBM CSLCs were isolated from a human glioblastoma surgical sample. (See Galli et al. Can Res. 2004; 64: 7011-7021) Glioblastoma CSLCs were maintained in their undifferentiated state using Neurobasal Media (Life Technologies GmbH, Frankfurt, Germany), supplemented with epidermal growth factor (10 μg/500 mL media) and fibroblastic growth factor (10 μg/500 mL media), sodium pyruvate, glutamine, B27, non-essential amino acids and penicillin/streptomycin (Gibco, Grand Island, N.Y.). GBM CSLCs displayed typical characteristics of stem cells as they grew as neurospheres, had increased expression of CD133 and nestin, and were highly tumorigenic after implantation in SCID-beige mice.

The anti-CTGF antibody, CLN-1, (FibroGen Inc., San Francisco, Calif., USA), was reconstituted in water (30 μg/ml) and stored at 4° C. Cells treated with both the anti-CTGF antibody and X-rays, were first exposed to the anti-CTGF antibody for two hours prior to irradiation with 6 MV X-rays (Mevatron, Siemens, Erlangen, Germany) at a dose rate of 2.5 Gy/min.

### Example 2

### Inhibition of Glioma Cells Migratory Ability with Anti-CTGF Antibody Treatment

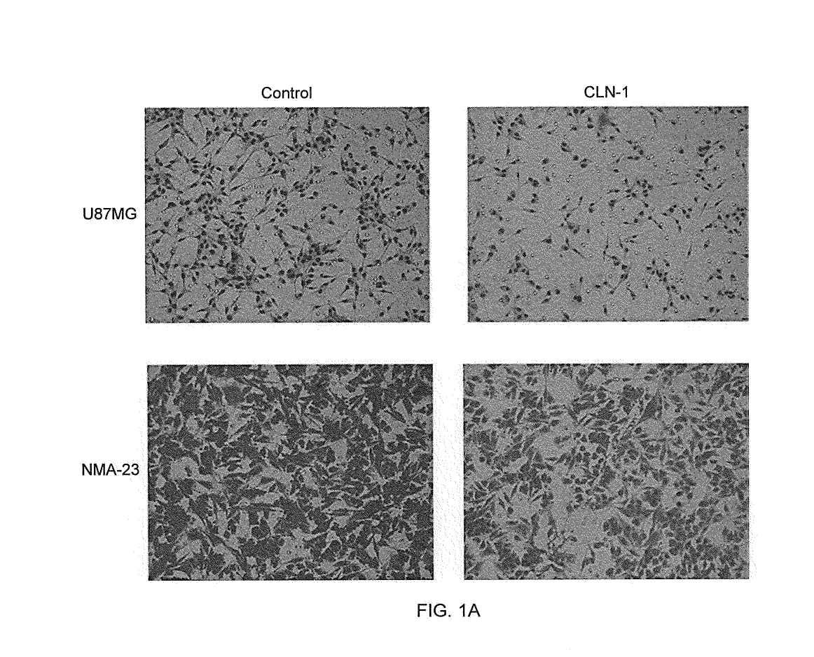

A transwell migration assay was used to study the effect of the anti-CTGF antibody (CLN-1, 30 μg/ml) and/or 4 Gy irradiation on the migration ability of U87MG and NMA-23 CSLCs through a collagen IV coated membrane (Life Technologies GmbH). The NMA-23 cells required subculturing in DMEM media (Life Technologies GmbH) with 10% FCS prior to the experiment in order for them adhere to the surface of the flasks. The media was removed and replaced with fresh DMEM media without any additives to starve the cells for 24 hours. This was followed by incubation with/without the anti-CTGF antibody for 2 hours. Specific flasks were then irradiated with 4 Gy. After treatment, cells were trypsinized and then plated onto the upper chamber of the transwells (10,000 cells in 50 μl/well). The lower chamber was previously filled with 280 μl of DMEM media containing 10% BSA. After 6 hours, the collagen IV coated membrane was removed and cells on the upper side were mechanically removed. The lower side of the membrane was stained with a Diff-quik staining system (Andwin Scientific, Schaumburg, Ill.). Representative photos (FIG. 1) were taken at 20× magnification and then the migrated cells were counted. These images in FIG. 1 demonstrates that treatment with an anti-CTGF antibody (CLN-1), irradiation or the combination of the anti-CTGF antibody with irradiation inhibit the in vitro migratory ability of U87MG and NMA-23 cells. Less U87MG and NMA-23 cells are apparent in the respective anti-CTGF antibody, irradiated and combined treatment wells compared to control.

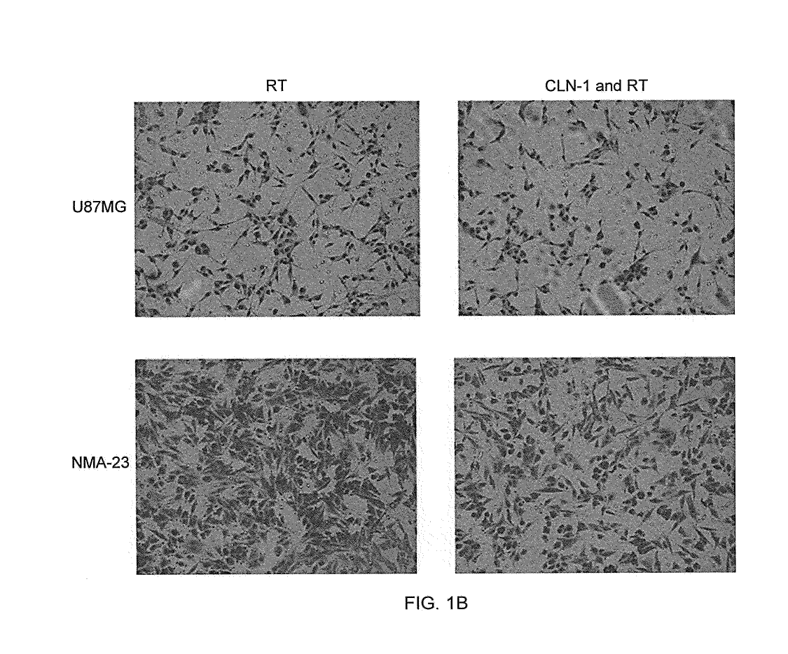

The change in migratory ability of U87MG and NMA-23 cells following treatment with an anti-CTGF antibody (CLN-1), irradiation or the combination of the anti-CTGF antibody with irradiation is illustrated quantitatively in FIGS. 2A and 2B. Irradiation did not significantly inhibit the relative number of U87MG cells that migrated (94.46±6.64% of control, p-value>0.05). Treatment with the anti-CTGF antibody, however, significantly inhibited migration of U87MG cells (44.60±5.75%, p-value<0.05) as did the combined treatment of anti-CTGF antibody and irradiation (60.53±1.44%, p-value<0.05). Similarly, irradiation did not significantly inhibit the relative number of NMA-23 cells that migrated (97.77±8.34%, p-value>0.05). Treatment with the anti-CTGF antibody, again, significantly inhibited migration of NMA-23 cells (71.91±5.52%, p-value<0.05) as did the combined treatment of anti-CTGF antibody and irradiation (68.06±2.95%, p-value<0.05). The qualitative and quantitative assay results demonstrate that treatment with an anti-CTGF agent can significantly decrease the migratory ability of glioma cells. A decrease in migratory ability may allow the use of an anti-CTGF agent as a neoadjuvant to decrease tumor cell invasion into surrounding normal tissue and also to improve surgical margins.

### Example 3

### Anti-CTGF Antibody Treatment Decreases Clonogenicity of Glioma Cell Lines

The effect of an anti-CTGF antibody on clonogenicity of two glioma cell lines, U87MG and T98, was tested by plating increasing numbers of cells (10³ to 8×10³) in 25 cm² flasks (Becton Dickinson, Heidelberg, Germany). Subsets were treated with an anti-CTGF antibody (CLN-1, 30 μg/ml) for 2 h. Further subsets were then irradiated (4 Gy). The cells were allowed to grow for 10-14 days before being fixed and then stained with crystal violet (Sigma, Germany). The flasks were examined using a microscope and only colonies with at least 50 cells were counted.

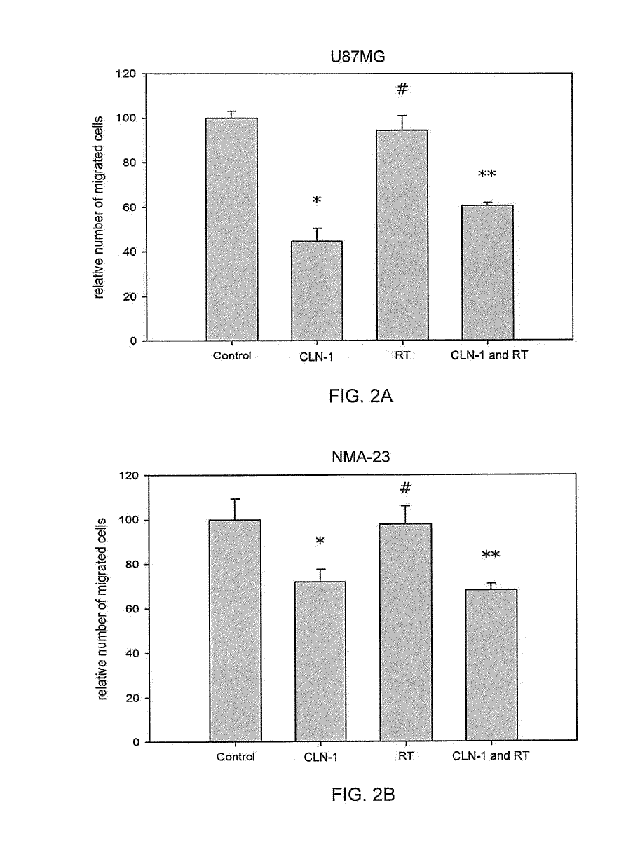

Treatment of U87MG cells with the anti-CTGF antibody (CLN-1, 30 μg/ml) decreased the number of clones to 66.95±3.11% (p-value<0.05) of control. FIG. 3A. Irradiation decreased the number of clones to 11.86±0.98% (p-value<0.05) of control. The combined modalities decreased the number of clones to 6.89±2.13% (p-value<0.05) of control.

Treatment of T98G cells with an anti-CTGF antibody (CLN-1, 30 μg/ml) decreased the number of clones 71.43±10.04% (p-value<0.05) of control. FIG. 3B. Irradiation decreased the number of clones to 37.37±2.32% (p-value<0.05) of control. The combined modalities decreased the number of clones to 25.86±1.98% (p-value<0.05) of control. The results demonstrate that treatment with an anti-CTGF agent decreases the ability of brain tumor cell lines to proliferate indefinitely, i.e., results in reproductive death.

### Example 4

### Anti-CTGF Antibody Treatment Decreases Proliferation Rate of Glioma Cell Lines

The effect of an anti-CTGF antibody on proliferation of two glioma cell lines, U87MG and T98, was tested by plating 5×10⁴ cells in 25 cm² flasks (Becton Dickinson) that were incubated grow overnight before being treated with an anti-CTGF antibody (CLN-1, 30 μg/ml) for 2 h, irradiated (4 Gy) or combined treatment with antibody and irradiation. The cells were incubated for 72 hours and then live cells were counted.

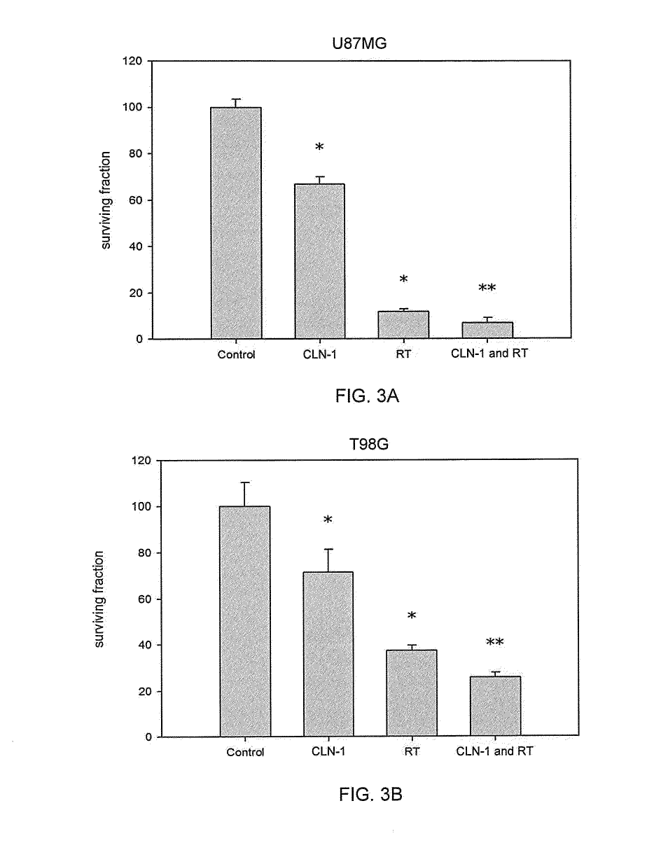

Treatment with an anti-CTGF antibody (CLN-1, 30 μg/ml) decreased U87MG cell number to 59.24±14.05% (p-value<0.05) of control. FIG. 4A. Irradiation decreased cell number to 57.76±2.34% (p-value<0.05) of control, while the combined treatment decreased the cell number to 45.21±6.46% (p-value<0.05) of control.

Treatment with an anti-CTGF antibody (CLN-1, 30 μg/ml) decreased T98G cells to 58.65μ6.31% (p-value<0.05) of control. FIG. 4B Irradiation decreased cell number to 57.93±6.35% (p-value<0.05) of control, while the combined treatment decreased the cell number to 36.42±0.36% (p-value<0.05). The results demonstrate that treatment with an anti-CTGF agent decreases the ability of brain tumor cell lines to proliferate by at least 40% compared to control. Further the decrease in proliferation rate achieved with the anti-CTGF agent appears to be at least additive to that achieved with radiation alone.

### Example 5

### Anti-CTGF Antibody Treatment Decreases Clonogenicity and Proliferation of GBM CSLCs In Vitro

Patient-derived GBM CSLCs spontaneously form neurospheres when cultured in NBE medium. Neurospheres are clonal balls of proliferating cells, each generated by a single-cell and serve as a hallmark of GBM stem cells. The ability to form neurospheres is also associated with worse clinical outcomes. Neurosphere formation can be used as a basis for a clonogenic assay. This was accomplished by first seeding 1,000 GBM CSLCs (NMA-23) into 25 cm² flasks. The flasks were then treated with an anti-CTGF antibody (CLN-1, 30 g/ml), irradiation (4 Gy) or the combined treatment. After 7 days incubation, the number of neurospheres in each flask were counted (10× magnification) and normalized for the number of neurospheres in control flasks.

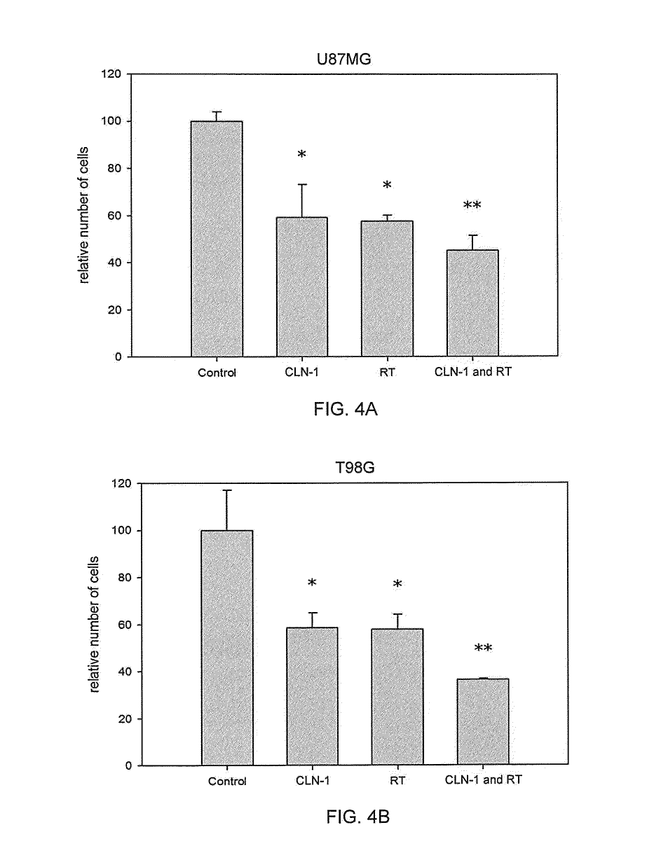

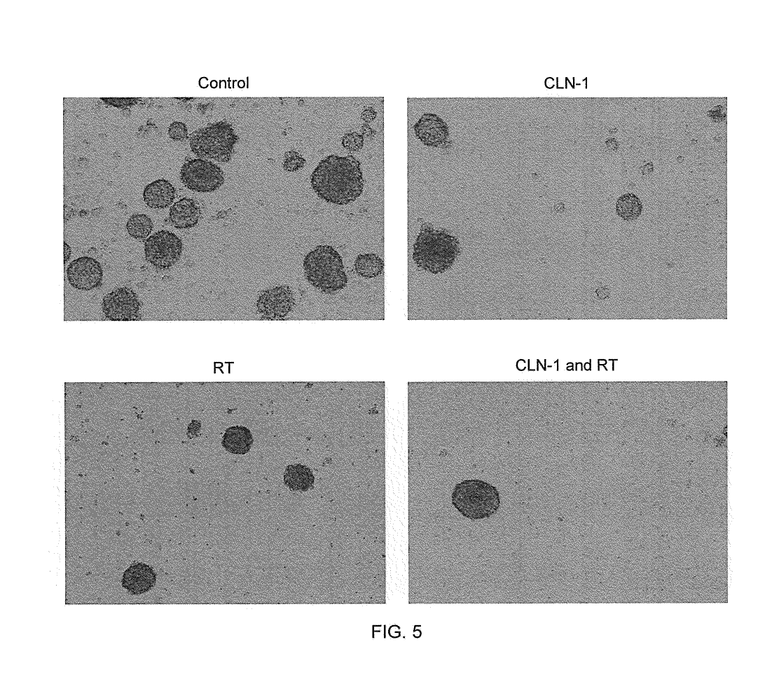

Representative fields from the different treatment groups are shown in FIG. 5 (10×) and relative number of spheres presented in FIG. 6. As can be seen, treatment with an anti-CTGF antibody (CLN-1, 30 μg/ml), irradiation (4 Gy), or the combined treatment decreased neurosphere formation to 60.52±12.53% (p-value<0.05), 30.62±2.52% (p-value<0.05), and 21.58±1.38 Z % (p-value<0.05), respectively, compared to control.

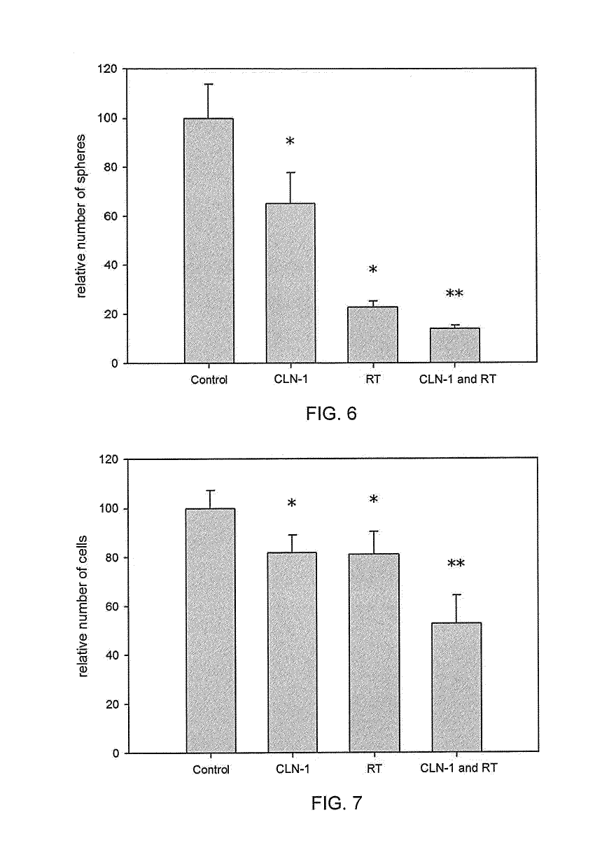

A proliferation assay was also performed on the NMA-23 GBM CSLC cells following the same conditions as detailed in Example 4. Treatment with an anti-CTGF antibody (CLN-1, 30 μg/ml), decreased the number of NMA-23 cells to 81.92±7.09% of control (p-value<0.05). FIG. 7. Irradiation decreased cell number to 81.21±9.11% of control (p-value<0.05), while the combined treatment of anti-CTGF antibody and irradiation decreased the cell number to 52.64±11.51% of control (p-value<0.05).

These results demonstrate that treatment with an anti-CTGF agent decreases the self-renewal capacity and proliferation rate of GBM CSLC cells by at least 15% compared to controls. Further, the decrease in self-renewal capacity and decrease in proliferation rate by an anti-CTGF agent is at least additive to that achieved with radiation.

### Example 6

### Anti-CTGF Antibody Treatment Decreases Self-Renewal Capacity of GBM CSLCs In Vitro

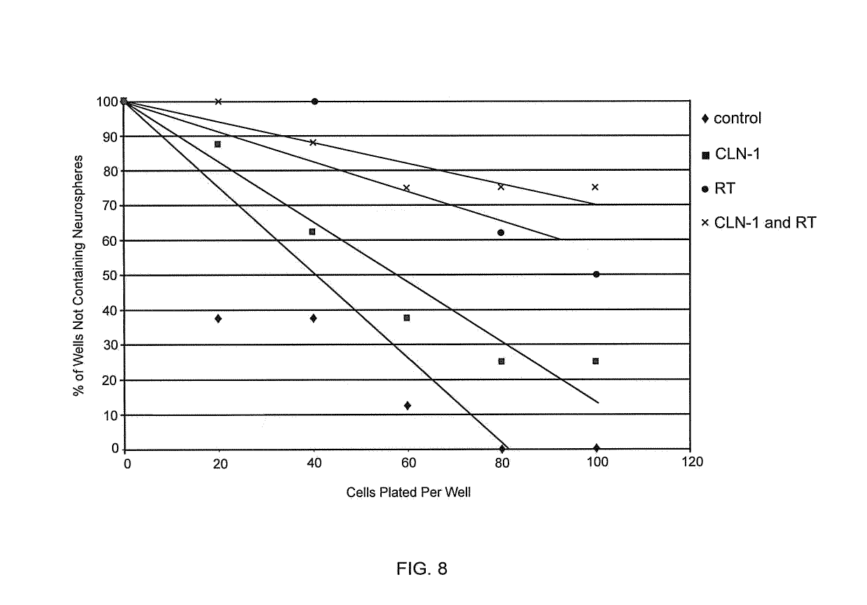

To measure the effect on an anti-CTGF antibody on GBM CSLC stem cell proliferation, a limiting dilution assay was performed by plating increasing numbers of GBM CSLCs (NMA-23) into wells of 96-well plates following treatment with an anti-CTGF antibody (CLN-1, 30 μg/ml), irradiation (4 Gy), or the combined treatment. After incubating the plates for 7 days, the number of neurospheres in each well were counted. Then the percentage of wells not containing neurospheres for each cell-plating density was calculated and plotted (Y axis) against the number of cells plated per well (X axis) to yield FIG. 8. With control (untreated) cells, all wells contained neurospheres once the plating density was greater than 80 cells/well. Treatment with CLN-1 (30 μg/ml) decreased the self-renewal capacity of NMA-23 cells such that when 80-100 cells were plated per well, 25% of the wells produced no neurospheres. Irradiation (4 Gy) resulted in 50-60% of the wells containing no neurospheres, while the combination of irradiation with the anti-CTGF antibody further decreased the self-renewal capacity of these CSLCs such that 75% of wells contained no neurospheres 7 days after 80-100 cells were plated in each well. The results demonstrate that treatment with an anti-CTGF agent decreases the proliferation rate and self-renewal capacity of GBM CSLCs.

### Example 7

### Anti-CTGF Antibody Treatment Inhibits Glioblastoma Growth in Mice

Beige SCID mice (8-12 wk old, 20 g; Charles River Laboratories, Sulzfeld, Germany) were anesthetized and stereotactically inoculated with GBM CSLC cells (NMA-23, 10⁴ cells in 2 μl PBS) via a 10 μl Hamilton syringe into the left forebrain (2 mm lateral to bregma, at a 3 mm depth from the skull surface) using a small animal stereotactic frame (World Precision Instruments, Germany GmbH, Berlin, Germany). The day following tumor inoculation, animals were randomized into four treatment groups: control, anti-CTGF antibody, irradiation, or the combined treatment of anti-CTGF antibody and irradiation (n=13-16, with three mice scheduled for histological examination). Five days after tumor inoculation, the entire head of anesthetized mice was irradiated with 7Gy as a single dose using a 6 MV LINAC (Siemens, Germany). Anti-CTGF antibody treatment (CLN-1, 3 mg/kg i.p. every other day) was initiated directly after irradiation and continued until the end of observation.

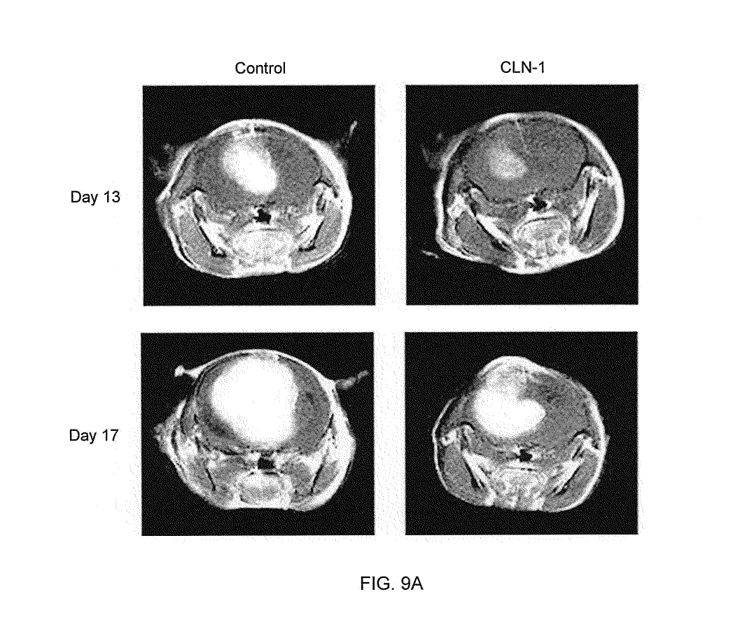

On days 13 and 17, animals were examined by magnetic resonance imaging (MRI) on a clinical 1.5-T whole-body MRI system (Siemens Magnetom Vision, Erlangen, Germany) using a custom-made small animal solenoid Tx/Rx radiofrequency coil. Tumor volumes were estimated using Gadolinium enhanced T1 weighted spin-echo images. FIGS. 9A and 9B show representative MRI images of SCID-beige mouse brains taken on day 13 (top row) and day 17 (bottom row) post-implantation of NMA-23 cells. Untreated animals appear to have larger and denser tumors on day 13 post-implantation compared to the tumors in treated mice. At day 17 post-implantation, the tumor of the control mouse continued to increase in size at a faster rate compared to the treated tumors.

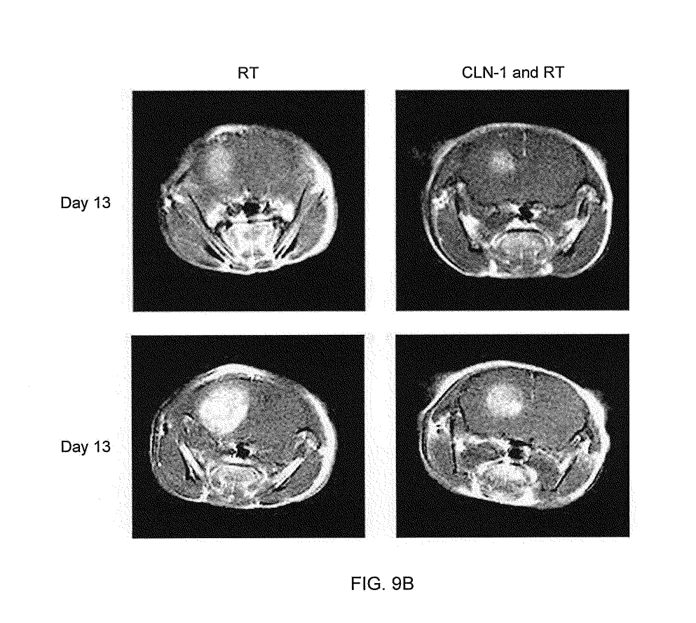

Three-dimensional reconstructions of tumor surfaces were performed. The tumor volumes were calculated using the formula v=length×width×width×0.5. The change in tumor volume following treatment is illustrated in FIG. 10. At day 13 post-implantation, differences were observed between the different treatments, but significance was not achieved. At day 17 post-implantation, the difference in tumor volume became significant with control tumors having a mean volume of 165.7±49.47 mm³, tumors treated with the anti-CTGF antibody had a mean volume of 93.83±23.27 mm³ (p-value<0.05), irradiated tumors had a mean volume of 84.53±15.89 mm³ (p-value<0.05), while the combined treatment tumors had a tumor volume of 31.83±15.15 mm³ (p-value<0.05). These data demonstrate that administration of an anti-CTGF agent inhibits brain tumor growth by at least 40% compared to untreated animals. Further the growth inhibition achieved with the anti-CTGF agent is at least additive to that achieved with radiation alone.

### Example 8

### Anti-CTGF Antibody Treatment Increases Survival of Mice Bearing Glioblastoma Xenografts

The ability of an anti-CTGF antibody (CLN-1), alone or with irradiation, to increase survival of SCID-beige mice orthotopically implanted with NMA-23 cells was studied. After tumor cell implantation mice randomized into 4 groups and treated as above in Example 7. Mice were observed daily and moribund mice or mice with severe neurologic symptoms were euthanized.

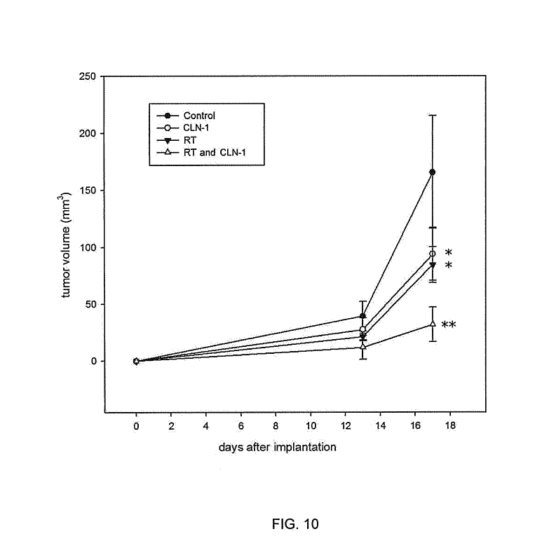

A Kaplan—Meier survival curve was generated, FIG. 11, that demonstrates that treatment with an anti-CTGF antibody can extend survival approximately 3 days compared to the untreated control mice, 20.89±0.79 days vs. 17.89±0.45 days (P<0.05, log-rank). Irradiation extended survival by approximately 4 days compared to control mice, 22.13±0.72 days vs. 17.89±0.45 days (P<0.05, log-rank). The combined treatment prolonged survival by approximately 6 days compared to control mice, to 23.88±1.01 days vs. 17.89±0.45 days (P<0.05, log-rank).

The survival curve results demonstrate that treatment with an anti-CTGF agent increases survival of mice with brain tumors by at least 15% compared to untreated animals. Moreover, the addition of an anti-CTGF agent to radiation further increased survival of mice with brain tumors an additional 10% compared to radiation alone.

### Example 9

### Histological and Immunohistological Evaluation of Representative Tumor and Normal Brain Tissue Specimens Following Treatment with Anti-CTGF Antibody and/or Irradiation

For histological and immunohistological analysis, three animals from each treatment group from Example 7 were sacrificed at day 18 by administration of a lethal dose of pentobarbital and were exsanguinated by transcardial perfusion first with ice-cold PBS (pH7.4, 10 ml/mouse). Brains were dissected out of the skull and either placed into formalin for preparation of paraffin-embedded slices or snap frozen and stored at −80° C. Frozen sections (20 μm thick transversal sections) were prepared on a cryomicrotome and placed on poly-L-Lysine-coated slides.

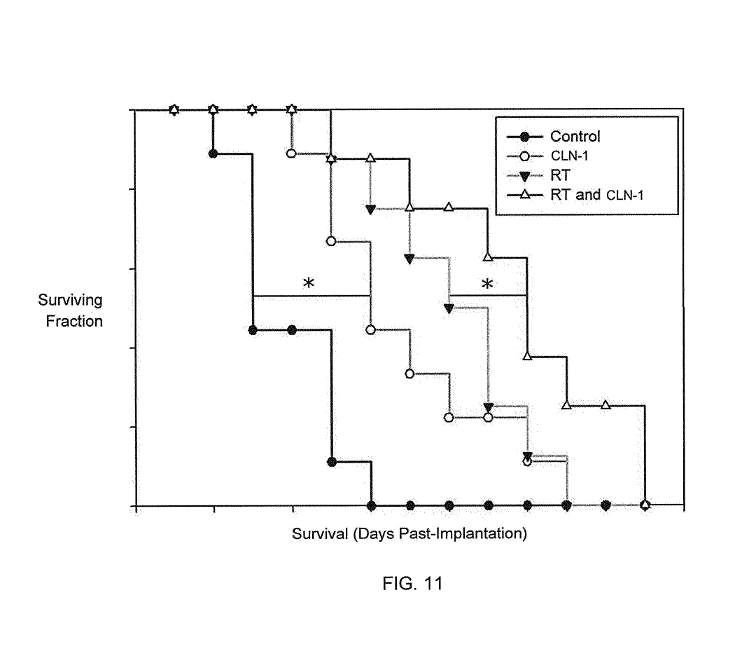

Paraffin-embedded slices were stained with hematoxylin and eosin (HE) and then examined using at 10× magnification to assess the degree of tumor cell infiltration into the surrounding brain parenchyma. Untreated tumors displayed modest infiltration of the brain that was frequently organized in perivascular cell clusters. FIG. 12. Anti-CTGF antibody treatment (CLN-1, 3 mg/kg i.p., every 2 days starting 6 days post-implantation) inhibits the invasion of brain parenchyma and produced sharp, well defined tumor margins. FIG. 12. In contrast, irradiation (7 Gy, 5 days post-implantation) produced highly diffuse tumor infiltration and extensions into the surrounding normal tissue that included single invading cells. FIG. 12. Additionally, the tumor margins of irradiated tumors were highly irregular and diffuse. The addition of an anti-CTGF antibody to radiation decreased the extent of tumor cell invasiveness and improved the tumor margin. FIG. 12. These data demonstrate the ability of an anti-CTGF agent to decrease normal and radiation-induced tumor cell motility and invasiveness. The decrease in tumor cell motility and invasiveness demonstrates the utility of an anti-CTGF agent to improve tumor margin. The ability to improve tumor margins should allow for cleaner surgical margins, reduced surgical margins and improved patient outcomes. The decrease in radiation-induced tumor cell motility and invasiveness should lead to increased survival of treated subjects compared to those treated with radiation alone.

Immunohistochemistry was carried out using standard methodologies. Collagen expression was detected using antibodies to collagen 4 and collagen 5A1 (rabbit polyclonal, Santa Cruz Biotechnology, Santa Cruz, Calif., USA 1:100). Proliferating cells were detected using an antibody to Ki-67 (Dako, Helsinki, Finland). Microvessel density was determined using an anti-CD-31 antibody (Becton-Dickinson, Franklin Lakes, N.J.). CTGF expression was detected using an antibody to CTGF (FibroGen Inc. San Francisco, Calif., USA). After the incubation with primary antibodies, the appropriate enzyme-conjugated or fluorescent-labeled secondary antibody was applied to the slides. Negative control slides were obtained by omitting the primary antibody. Images were captured using a Nikon Eclipse E600 microscope (Nikon GmbH, Düsseldorf, Germany) equipped with a Nikon digital sight DS-UI camera (Nikon) and subsequently analyzed using GNU Image Manipulation Program 2.0 (GIMP 2.0, available at the GIMP website). For each treatment condition, the analysis of tissues was done in at least 10 randomly chosen fields taken from 3 to 5 sections.

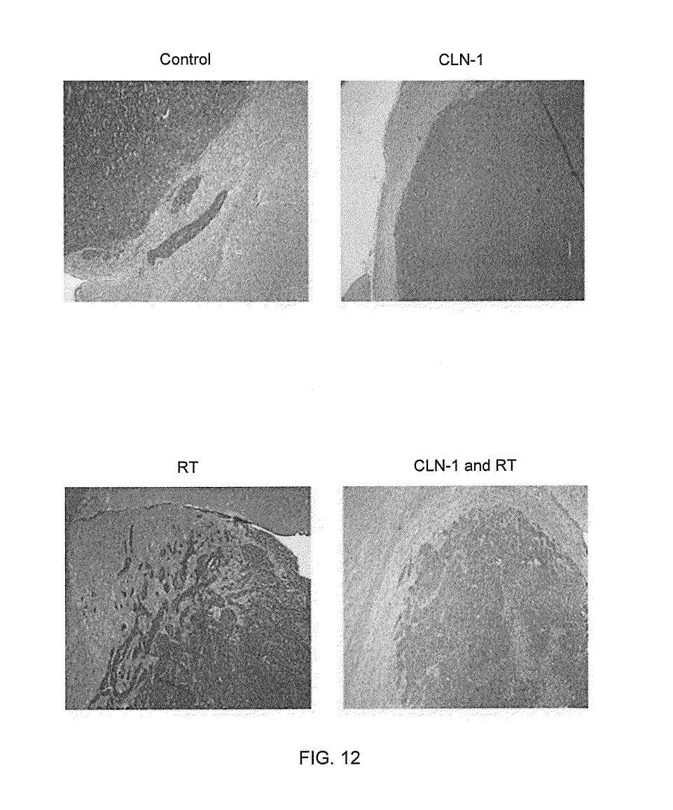

The percentage of tumor cells that were positive for intracellular CTGF was 86.97±7.6% in untreated mice. FIG. 13. Treatment with an anti-CTGF agent decreased the percentage of CTGF positive cells to 42.37±13.95%, p-value<0.05. The percentage of CTGF positive cells from irradiated mice was not significantly different from control mice, 84.11±7.59%, p-value>0.05. The addition of an anti-CTGF antibody to irradiation decreased the number of CTGF positive tumor cells to the level seen with anti-CTGF antibody alone, 42.99-11.0% CTGF positive cells, p-value<0.05. These data demonstrate that the administration of an anti-CTGF agent decreases the intracellular levels of CTGF in brain tumor cells.

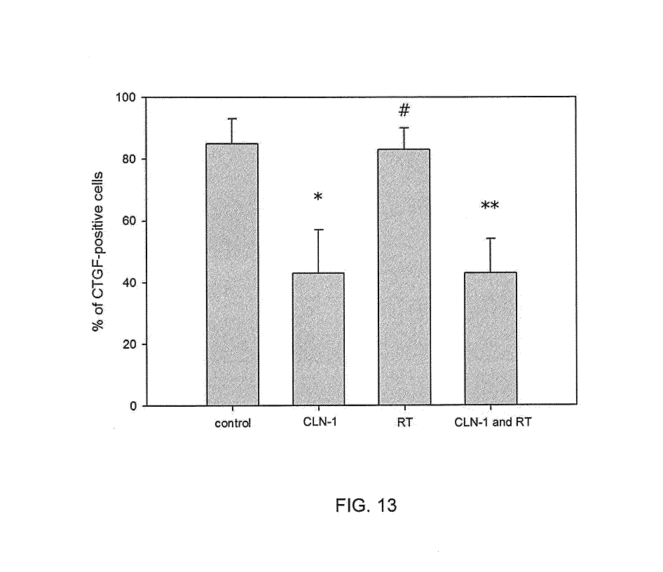

Treatment with anti-CTGF antibody decreased tumor cell proliferation as measured by the percentage of cells staining positive for Ki-67, with 20.81±3.64% (p-value<0.05) of the anti-CTGF antibody treated tumor cells staining positive for Ki-67, compared to untreated tumor cells, 53.74±4.1% Ki-67 positive cells. FIG. 14. Irradiation also decreased Ki-67 expression with 12.88±2.57% (p-value<0.05) of the cells expressing Ki-67. The combined treatment further decreased Ki-67 expression in tumor cells to 8.18±1.88% (p-value<0.05). These data demonstrate that the administration of an anti-CTGF agent decreases the expression of a marker of tumor cell proliferation.

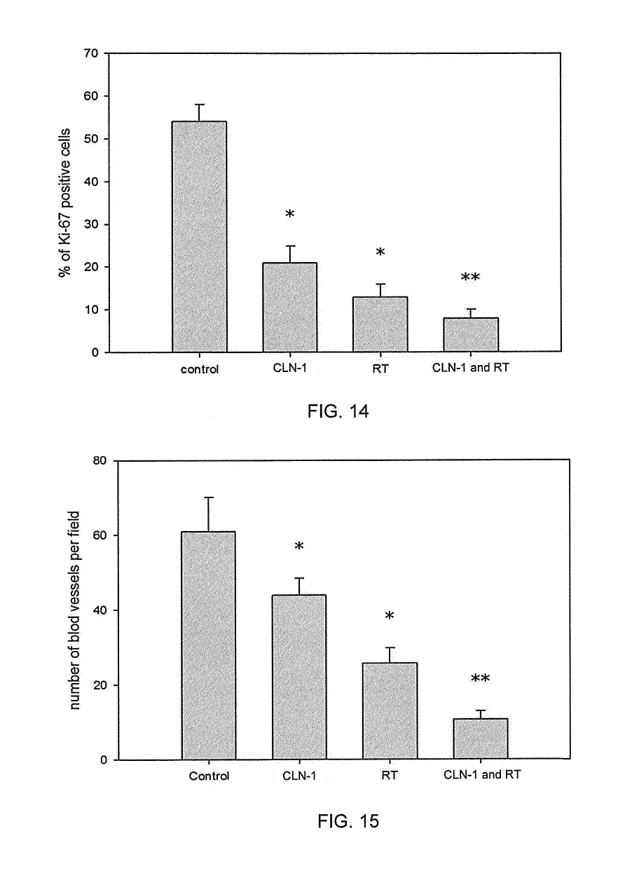

Tumor vascularization was decreased in tumor cells treated with an anti-CTGF antibody as measured by the percentage of CD-31 positive cells. CD-31 is a marker for endothelial cells that comprise tumor vasculature. Control tumors had 60.88±9.25 microvessels per field. FIG. 15. Anti-CTGF antibody treated mice had decreased numbers of endothelial cells per field, 44.0±4.47, p-value<0.05. Irradiation also decreased the number of microvessel per field, 25.75±4.03, p-value<0.05. The combined treatment of anti-CTGF antibody and irradiation further decreased microvessel density per field to 10.6±2.3, p-value<0.05. These data demonstrate that the administration of an anti-CTGF agent decreases the vascularity of a brain tumor.

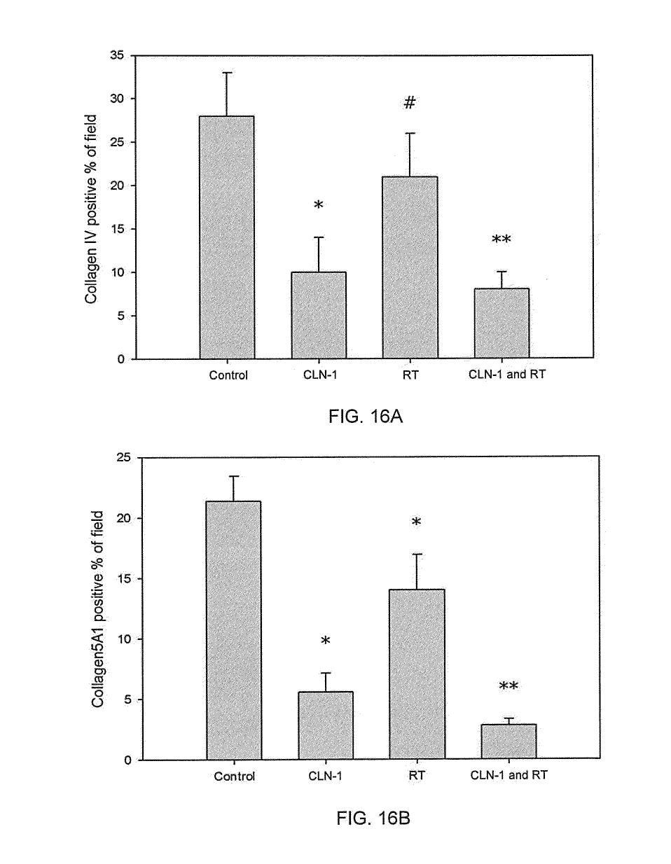

Collagen deposition was decreased in tumors treated with anti-CTGF antibody. The percentage of tumor tissue that was positive for collagen IV in untreated tumors was 28.28±4.84%. FIG. 16A. Treatment with an anti-CTGF antibody significantly decreased the percentage of collagen IV positive area to 10.17±4.12% (p-value<0.05). Irradiation did not significantly decrease the percentage of tumor tissue that was positive for collagen IV expression, 20.53±5.44%, p-value>0.5. The combined anti-CTGF antibody and irradiation treatment significantly decreased the percentage of tumor tissue that was positive for collagen expression to 8.23±2.01%, p value<0.05. These data demonstrate that administration of an anti-CTGF agent decreases tumor-associated extracellular matrix deposition.

Similarly, the percentage of tumor tissue that was positive for collagen VA1 in untreated tumors was 21.34±2.06%. FIG. 16B. Treatment with an anti-CTGF antibody significantly decreased the percentage of tumor tissue that was collagen VA1 positive to 5.6±1.52%, p-value<0.05. Irradiation also decreased the percentage of tumor tissue positive for collagen VA1 expression to 13.99±2.92%, p-value<0.05. The combined therapy of anti-CTGF antibody and irradiation further decreased the percentage of tumor tissue that was collagen VA1 positive area to 2.8±0.54%, p-value<0.05. These data again demonstrate that the administration of an anti-CTGF agent decreases the deposition of tumor-associated extracellular matrix, a factor that may be particularly useful in the treatment of desmoplastic tumors, including medullblastomas, and desmoplastic infantile and noninfantile gangliomas.

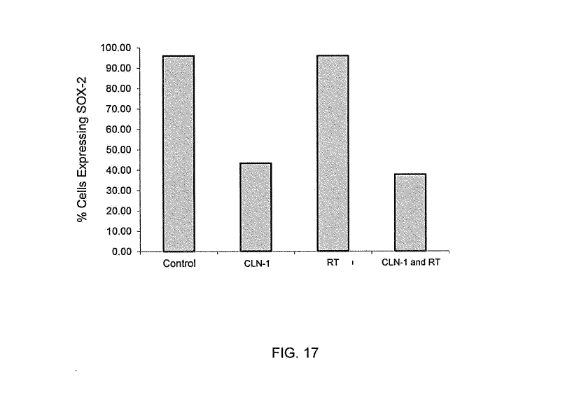

Treatment with an anti-CTGF antibody also decreased expression of the stem cell marker SOX-2. The number of tumor cells expressing SOX-2 was determined by immunofluorescence. FIG. 17. In tumors from control mice, about 96% of tumor cells expressed SOX-2. Treatment with the anti-CTGF antibody (CLN-1) decreased the expression of SOX-2 such that only about 43% of tumor cells expressed the stem cell marker. Irradiation had no impact on SOX-2 expression and about 96% of cells in tumors of mice treated with irradiation expressed SOX-2 in the absence of CLN-1, while only about 38% of tumor cells expressed SOX-2 in mice that were irradiated and treated with the anti-CTGF antibody. These data demonstrate that treatment with an anti-CTGF agent induces tumor cell differentiation. The induction of tumor cell differentiation should decrease the rate of tumor cell proliferation. Further, an anti-CTGF agent may be used with other differentiation inducing agents, such as retinoic acid, to increase the degree of tumor cell differentiation. Combination therapy may further extend patient survival. Increased glioma differentiation can be measured by assaying for up-regulation of GFAP.

### Example 10

### Anti-CTGF Antibody Treatment Inhibits Migratory Ability of Metastatic Breast Cancer Cells

A transwell migration assay is set up as in Example 2 using a metastatic breast cancer cell line (231BR, a brain tropic subline of the human MDA-MB-231, Dr. Yoneda, University of Texas at San Antonio, San Antonio, Tex.). Treatment with an anti-CTGF antibody (CLN-1) decreases the migration rate of breast cancer cells. Further, anti-CTGF antibody treatment decreases radiation-induced migration.

### Example 11

### Anti-CTGF Antisense Oligonucleotide Inhibits Migratory Ability of Glioma Cells

A transwell migration assay is set up as in Example 2, but instead of treating the glioma cells with an anti-CTGF antibody, an anti-CTGF antisense oligonucleotide is used. Treatment with a scrambled antisense oligonucleotide serves as the control. Cells treated with the anti-CTGF antisense oligonucleotide have decreased glioma cell migration compared to the glioma cells treated with scrambled antisense oligonucleotide. Treatment with anti-CTGF antisense oligonucleotide also inhibits the radiation-induced migration of glioma cells.

### Example 12

### Anti-CTGF Antisense Oligonucleotide Treatment Decreases Clonogenicity and Proliferation of Glioma Cell Lines

For clonogenic survival experiments, subconfluent U87MG cells are cells treated with anti-CTGF antisense oligonucleotides or scrambled antisense oligonucleotides at a fixed concentration for 24 h. Cells are irradiated with 0, 2, 4, 6 or 8 Gy and then trypsinized and plated at appropriate predetermined dilutions onto 5-cm culture dishes. In general, between 200 and 2000 cells are seeded per dish in order to obtain between 100 and 300 single well-separated colonies per dish. After 8 to 12 days, cell culture plates are washed, fixed in 95% ethanol and stained with 0.1% crystal violet. Colonies containing more than 50 cells are scored as clonogenic survivors. The colony-forming experiments are performed three times for each treatment.

The dose-response analysis of survival in the presence of a fixed oligonucleotide concentration and increasing doses of irradiation demonstrates a decline of survival (measured in percent of untreated controls) after 0, 2, 4, 6 and 8 Gy irradiation alone. In the presence of an anti-CTGF antisense oligonucleotide survival is further decreased in a manner that increases with increasing radiation dose. The scrambled antisense oligonucleotide produces a less marked decrease of survival as compared to the anti-CTGF antisense oligonucleotide.

### Example 13

### Anti-CTGF Antisense Oligonucleotide Treatment Increases Survival of Mice Bearing Glioblastoma Xenografts

NMA-23 neurospheres are implanted orthotopically into the striata of SCID-beige mice. The mice are randomized and placed into four treatment groups. Group 1 is the control group and remains untreated. Starting six days post-implantation and continuing every 2 days, group 2 is treated with anti-CTGF antisense oligonucleotide as a single stereotaxic injection (60 μl, 0.2 μM). Group 3 is irradiated with 7 Gy on day 6 post-implantation. Group 4 receives the combined anti-CTGF antisense oligonucleotide treatment and 7 Gy irradiation.

The mice are monitored twice a day for overall condition. Mice in obvious pain, unresponsive to touch or other stimuli and those with labored breathing are euthanized. Group 1 (control) mice survive for 18 days. Group 2 mice (anti-CTGF antisense oligonucleotide) survive for 21 days. Group 3 mice (irradiated) survive for 22 days. Group 4 (combined treatment) mice survive for 25 days. The experiment demonstrates the effectiveness of anti-CTGF oligonucleotide therapy in extending the survival of mice bearing glioblastoma xenografts.

### Example 14

### Neoadjuvant Treatment of Human Glioblastoma with an Anti-CTGF Antibody

A patient diagnosed with a glioblastoma is administered a therapeutically effective amount of an anti-CTGF antibody immediately upon diagnosis of the disease. The patient is then scheduled for surgery. The patient undergoes conventional cranial surgery where the surgeon resects as much of the tumor, preferably the entire tumor, as is possible. During surgery, the surgeon notices that the tumor margin is more organized than is typical for glioblastomas and as a result, the tumor margin is narrower than is typical for a glioblastoma allowing the surgeon to remove less adjacent normal brain tissue.

Tumor specimens including regions of the tumor margin and adjoining normal tissue are analyzed histologically where it is confirmed that neoadjuvant treatment with an anti-CTGF antibody produces a more organized and uniform tumor margin than is typical seen in glioblastomas. There are less tumor extensions into the normal tissue. Further, the normal tissue adjoining the tumor margin shows a decreased number of invasive glioblastoma cells and those tumor cells that have in fact invaded the normal tissue have not migrated as far as one would normally expect. Additionally, it is noted that the microvessel density within the tumor sections are decreased as is the level of extracellular matrix components including collagen type IV and VAI. Lower microvessel density and lower levels of extracellular matrix deposition are both associated with improved prognosis.

Following surgery, the patient is treated with conventional radiation therapy and temozolmide chemotherapy, plus an anti-CTGF antibody. The use of the anti-CTGF antibody as a neoadjuvant and the addition of the anti-CTGF antibody to the conventional standard of care treatment extends the patient's survival 6 months beyond the median survival of similarly situated patients that receive only standard therapy.

### Example 15

### Treatment of Human Glioblastoma with an Anti-CTGF Antisense Oligonucleotide

A patient with a glioblastoma is prepared for cranial surgery in a conventional manner. The surgeon performs a conventional craniotomy and resection of the tumor. The surgeon removes as much of the tumor as is possible, while minimizing any damage to adjacent tissue. Next, the surgeon places into to the resection site a therapeutically effective amount of compressed hollow foam beads loaded with an anti-CTGF antisense oligonucleotide. The surgeon ensures that there are sufficient foam beads to fill the entire resection cavity upon expansion of the beads. The beads adjacent to the tissue surrounding the cavity of the resection site will substantially conform to the contours of the cavity. The surgical site is then closed in a conventional manner.

Following surgery, the patient is treated with conventional radiation therapy and temozolmide chemotherapy. The addition of the anti-CTGF antisense oligonucleotide treatment to the conventional standard of care treatment extends the patient's overall survival 4 months beyond the median survival of similarly situated patients that received only standard therapy.

### Example 16

### Prevention of Recurrence of Low Grade Astrocytoma

A patient with a grade 1 astrocytoma is treated in the conventional manner by surgical resection of the tumor. The surgeon is able to totally resect the tumor. After recovering from surgery, the patient is administered prophylactically a therapeutically effective amount of an anti-CTGF agent on a regular basis to prevent tumor recurrence from residual tumor cells that either were shed during surgery or that had invaded into the surrounding, unexcised normal brain tissue prior to surgery. The patient is followed with regular radiographic studies including MRI. The prophylactic treatment with an anti-CTGF agent delays recurrence of the astrocytoma 2 years beyond the median time to disease recurrence seen in a control group of patients that only had their tumor surgically excised.

What is claimed:

1. A method for treating a brain tumor, the method comprising administering to a subject in need thereof an effective amount of an anti-CTGF agent, thereby treating the brain tumor.

2. The method of claim 1, wherein the brain tumor is a glioma.

3. The method of claim 2, wherein the glioma is an astrocytoma.

4. The method of claim 2, wherein the glioma is a glioblastoma.

5. The method of claim 1, wherein the brain tumor is a metastasis.

6. The method of claim 1, wherein the brain tumor is a recurrent tumor.

7. The method of claim 1, wherein the brain tumor is not substantially resectable.

8. The method of claim 1, wherein the anti-CTGF agent is an anti-CTGF antibody, anti-CTGF antibody fragment, anti-CTGF antibody mimetic or anti-CTGF oligonucleotide.

9. The method of claim 8, wherein the anti-CTGF agent is an anti-CTGF antibody.

10. The method of claim 9, wherein the anti-CTGF antibody is identical to CLN-1.

11. The method of claim 10, wherein the anti-CTGF antibody binds to the same epitope as CLN-1.

12. The method of claim 8, wherein the anti-CTGF oligonucleotide is an antisense oligonucleotide, siRNA, shRNA or miRNA.

13. The method of claim 1, further comprising the administration of an additional therapeutic modality selected from the group consisting of chemotherapy, radiotherapy, immunotherapy and surgery.

14. The method of claim 13, wherein chemotherapy comprises the administration of temozolmide, procarbazine, lomustine, vincristine, cisplatin, carmustine, carboplatin or methotrexate.

15. The method of claim 1, wherein the anti-CTGF agent is administered as a neoadjuvant.

16. The method of claim 1, wherein the administration of an effective amount of an anti-CTGF agent prolongs the survival of the patient.

17. The method of claim 16, wherein the prolongation of survival is the prolongation of survival of at least 2 weeks, at least 1 month, at least 2 months, at least 3 months, at least 4 months, at least 5 months, at least 6 months, at least 8 months, at least 10 months, at least 12 months, at least 15 months, at least 18 months or at least 24 months compared to a control group or historical group.

18. The method of claim 16, wherein the prolongation of survival is the prolongation of disease-free survival, progression-free survival or overall survival.

19. The method of claim 1, wherein treating comprises decreasing brain tumor cell invasiveness.

20. The method of claim 19, wherein the brain tumor cell invasiveness is radiation-induced invasiveness.

21. The method of claim 1, wherein treating comprises decreasing brain tumor cell proliferation, decreasing brain tumor vascularity, decreasing deposition of brain tumor extracellular matrix, decreasing intracellular levels of CTGF in brain tumor cells or reactive astrocytes, decreasing the intracellular level of a stem cell marker in brain tumor cells, or decreasing reactive astrocyte induction.

22. The method of claim 1, wherein treating comprises improving tumor surgical margin or increasing brain tumor cell differentiation.


SEQUENCE LISTING (183 sequences)

SEQ ID NO: 1 - protein, 349 aa, Homo sapiens
MTAASMGPVRVAFVVLLALCSRPAVGQNCSGPCRCPDEPAPRCPAGVSLVLDGCGCCRVCAKQLGELCTERDPCDPHKGLFCDFGSPANRKIGVCTAKDGAPCIFGGTVYRSGESFQSSCKYQCTCLDGAVGCMPLCSMDVRLPSPDCPFPRRVKLPGKCCEEWVCDEPKDQTVVGPALAAYRLEDTFGPDPTMIRANCLVQTTEWSACSKTCGMGISTRVTNDNASCRLEKQSRLCMVRPCEADLEENIKKGKKCIRTPKISKPIKFELSGCTSMKTYRAKFCGVCTDGRCCTPHRTTTLPVEFKCPDGEVMKKNMMFIKTCACHYNCPGDNDIFESLYYRKMYGDMA

SEQ ID NO: 2 - DNA, 20 bp, Artificial Sequence
  Antisense Oligonucleotide
aggcacgtgcactggtactt

SEQ ID NO: 3 - DNA, 20 bp, Artificial Sequence
  Antisense Oligonucleotide
ccaggcacgtgcactggtac

SEQ ID NO: 4 - DNA, 20 bp, Artificial Sequence
  Antisense Oligonucleotide
acgaacgtccatgctgcaca

SEQ ID NO: 5 - DNA, 20 bp, Artificial Sequence
  Antisense Oligonucleotide
cacacccactcctcgcagca

SEQ ID NO: 6 - DNA, 20 bp, Artificial Sequence
  Antisense Oligonucleotide
ggcaggcccaaccacggttt

SEQ ID NO: 7 - DNA, 20 bp, Artificial Sequence
  Antisense Oligonucleotide
gtgtcttccagtcggtaagc

SEQ ID NO: 8 - DNA, 20 bp, Artificial Sequence
  Antisense Oligonucleotide
accaggcagttggctctaat

SEQ ID NO: 9 - DNA, 20 bp, Artificial Sequence
  Antisense Oligonucleotide
ggaccaggcagttggctcta

SEQ ID NO: 10 - DNA, 20 bp, Artificial Sequence
  Antisense Oligonucleotide
ctggaccaggcagttggctc

SEQ ID NO: 11 - DNA, 20 bp, Artificial Sequence
  Antisense Oligonucleotide
gtctggaccaggcagttggc

SEQ ID NO: 12 - DNA, 20 bp, Artificial Sequence
  Antisense Oligonucleotide
gtcattggtaacccgggtgg

SEQ ID NO: 13 - DNA, 20 bp, Artificial Sequence
  Antisense Oligonucleotide
ttgtcattggtaacccgggt

SEQ ID NO: 14 - DNA, 20 bp, Artificial Sequence
  Antisense Oligonucleotide
ggcggctctgcttctctagc

SEQ ID NO: 15 - DNA, 20 bp, Artificial Sequence
  Antisense Oligonucleotide
gaccatgcacaggcggctct

SEQ ID NO: 16 - DNA, 20 bp, Artificial Sequence
  Antisense Oligonucleotide
caaggcctgaccatgcacag

SEQ ID NO: 17 - DNA, 20 bp, Artificial Sequence
  Antisense Oligonucleotide
ttcgcaaggcctgaccatgc

SEQ ID NO: 18 - DNA, 20 bp, Artificial Sequence
  Antisense Oligonucleotide
tgttctcttccaggtcagct

SEQ ID NO: 19 - DNA, 20 bp, Artificial Sequence
  Antisense Oligonucleotide
gcccttcttaatgttctctt

SEQ ID NO: 20 - DNA, 20 bp, Artificial Sequence
  Antisense Oligonucleotide
ttgcccttcttaatgttctc

SEQ ID NO: 21 - DNA, 20 bp, Artificial Sequence
  Antisense Oligonucleotide
ttttgcccttcttaatgttc

SEQ ID NO: 22 - DNA, 20 bp, Artificial Sequence
  Antisense Oligonucleotide
ctttttgcccttcttaatgt

SEQ ID NO: 23 - DNA, 20 bp, Artificial Sequence
  Antisense Oligonucleotide
cactttttgcccttcttaat

SEQ ID NO: 24 - DNA, 20 bp, Artificial Sequence
  Antisense Oligonucleotide
tgcactttttgcccttctta

SEQ ID NO: 25 - DNA, 20 bp, Artificial Sequence
  Antisense Oligonucleotide
gatgcactttttgcccttct

SEQ ID NO: 26 - DNA, 20 bp, Artificial Sequence
  Antisense Oligonucleotide
cggatgcactttttgccctt

SEQ ID NO: 27 - DNA, 20 bp, Artificial Sequence
  Recognizes human
tacggatgcactttttgccc

SEQ ID NO: 28 - DNA, 20 bp, Artificial Sequence
  Antisense Oligonucleotide
gagtacggatgcactttttg

SEQ ID NO: 29 - DNA, 20 bp, Artificial Sequence
  Antisense Oligonucleotide
gggagtacggatgcactttt

SEQ ID NO: 30 - DNA, 20 bp, Artificial Sequence
  Antisense Oligonucleotide
ttgggagtacggatgcactt

SEQ ID NO: 31 - DNA, 20 bp, Artificial Sequence
  Antisense Oligonucleotide
ggcttggagattttgggagt

SEQ ID NO: 32 - DNA, 20 bp, Artificial Sequence
  Antisense Oligonucleotide
taggcttggagattttggga

SEQ ID NO: 33 - DNA, 20 bp, Artificial Sequence
  Antisense Oligonucleotide
acttgataggcttggagatt

SEQ ID NO: 34 - DNA, 20 bp, Artificial Sequence
  Antisense Oligonucleotide
aaacttgataggcttggaga

SEQ ID NO: 35 - DNA, 20 bp, Artificial Sequence
  Antisense Oligonucleotide
gcagccagaaagctcaaact

SEQ ID NO: 36 - DNA, 20 bp, Artificial Sequence
  Antisense Oligonucleotide
gtgcagccagaaagctcaaa

SEQ ID NO: 37 - DNA, 20 bp, Artificial Sequence
  Antisense Oligonucleotide
ctggtgcagccagaaagctc

SEQ ID NO: 38 - DNA, 20 bp, Artificial Sequence
  Antisense Oligonucleotide
tgctggtgcagccagaaagc

SEQ ID NO: 39 - DNA, 20 bp, Artificial Sequence
  Antisense Oligonucleotide
catgctggtgcagccagaaa

SEQ ID NO: 40 - DNA, 20 bp, Artificial Sequence
  Antisense Oligonucleotide
ttcatgctggtgcagccaga

SEQ ID NO: 41 - DNA, 20 bp, Artificial Sequence
  Antisense Oligonucleotide
tcttcatgctggtgcagcca

SEQ ID NO: 42 - DNA, 20 bp, Artificial Sequence
  Antisense Oligonucleotide
tgtcttcatgctggtgcagc

SEQ ID NO: 43 - DNA, 20 bp, Artificial Sequence
  Antisense Oligonucleotide
tatgtcttcatgctggtgca

SEQ ID NO: 44 - DNA, 20 bp, Artificial Sequence
  Antisense Oligonucleotide
ggtatgtcttcatgctggtg

SEQ ID NO: 45 - DNA, 20 bp, Artificial Sequence
  Antisense Oligonucleotide
ttagctcggtatgtcttcat

SEQ ID NO: 46 - DNA, 20 bp, Artificial Sequence
  Antisense Oligonucleotide
atttagctcggtatgtcttc

SEQ ID NO: 47 - DNA, 20 bp, Artificial Sequence
  Antisense Oligonucleotide
gaatttagctcggtatgtct

SEQ ID NO: 48 - DNA, 20 bp, Artificial Sequence
  Antisense Oligonucleotide
ctccacagaatttagctcgg

SEQ ID NO: 49 - DNA, 20 bp, Artificial Sequence
  Antisense Oligonucleotide
gccgtcggtacatactccac

SEQ ID NO: 50 - DNA, 20 bp, Artificial Sequence
  Antisense Oligonucleotide
atcggccgtcggtacatact

SEQ ID NO: 51 - DNA, 20 bp, Artificial Sequence
  Antisense Oligonucleotide
agcatcggccgtcggtacat

SEQ ID NO: 52 - DNA, 20 bp, Artificial Sequence
  Antisense Oligonucleotide
gcagcatcggccgtcggtac

SEQ ID NO: 53 - DNA, 20 bp, Artificial Sequence
  Antisense Oligonucleotide
gtgcagcatcggccgtcggt

SEQ ID NO: 54 - DNA, 20 bp, Artificial Sequence
  Antisense Oligonucleotide
gggtgcagcatcggccgtcg

SEQ ID NO: 55 - DNA, 20 bp, Artificial Sequence
  Antisense Oligonucleotide
cacttgaactccaccggcag

SEQ ID NO: 56 - DNA, 20 bp, Artificial Sequence
  Antisense Oligonucleotide
ggcacttgaactccaccggc

SEQ ID NO: 57 - DNA, 20 bp, Artificial Sequence
  Antisense Oligonucleotide
agggcacttgaactccaccg

SEQ ID NO: 58 - DNA, 20 bp, Artificial Sequence
  Antisense Oligonucleotide
tcagggcacttgaactccac

SEQ ID NO: 59 - DNA, 20 bp, Artificial Sequence
  Antisense Oligonucleotide
ccgtcagggcacttgaactc

SEQ ID NO: 60 - DNA, 20 bp, Artificial Sequence
  Antisense Oligonucleotide
cgccgtcagggcacttgaac

SEQ ID NO: 61 - DNA, 20 bp, Artificial Sequence
  Antisense Oligonucleotide
ctcgccgtcagggcacttga

SEQ ID NO: 62 - DNA, 20 bp, Artificial Sequence
  Antisense Oligonucleotide
acctcgccgtcagggcactt

SEQ ID NO: 63 - DNA, 20 bp, Artificial Sequence
  Antisense Oligonucleotide
tgacctcgccgtcagggcac

SEQ ID NO: 64 - DNA, 20 bp, Artificial Sequence
  Antisense Oligonucleotide
catgacctcgccgtcagggc

SEQ ID NO: 65 - DNA, 20 bp, Artificial Sequence
  Antisense Oligonucleotide
cttcttcatgacctcgccgt

SEQ ID NO: 66 - DNA, 20 bp, Artificial Sequence
  Antisense Oligonucleotide
ttcttcttcatgacctcgcc

SEQ ID NO: 67 - DNA, 20 bp, Artificial Sequence
  Antisense Oligonucleotide
ggcacaggtcttgatgaaca

SEQ ID NO: 68 - DNA, 20 bp, Artificial Sequence
  Antisense Oligonucleotide
tggcaggcacaggtcttgat

SEQ ID NO: 69 - DNA, 20 bp, Artificial Sequence
  Antisense Oligonucleotide
aatggcaggcacaggtcttg

SEQ ID NO: 70 - DNA, 20 bp, Artificial Sequence
  Antisense Oligonucleotide
gtaatggcaggcacaggtct

SEQ ID NO: 71 - DNA, 20 bp, Artificial Sequence
  Antisense Oligonucleotide
ttgtaatggcaggcacaggt

SEQ ID NO: 72 - DNA, 20 bp, Artificial Sequence
  Antisense Oligonucleotide
agttgtaatggcaggcacag

SEQ ID NO: 73 - DNA, 20 bp, Artificial Sequence
  Antisense Oligonucleotide
gacagttgtaatggcaggca

SEQ ID NO: 74 - DNA, 20 bp, Artificial Sequence
  Antisense Oligonucleotide
ccgggacagttgtaatggca

SEQ ID NO: 75 - DNA, 20 bp, Artificial Sequence
  Antisense Oligonucleotide
ctccgggacagttgtaatgg

SEQ ID NO: 76 - DNA, 20 bp, Artificial Sequence
  Antisense Oligonucleotide
gtctccgggacagttgtaat

SEQ ID NO: 77 - DNA, 20 bp, Artificial Sequence
  Antisense Oligonucleotide
ttgtctccgggacagttgta

SEQ ID NO: 78 - DNA, 20 bp, Artificial Sequence
  Antisense Oligonucleotide
cattgtctccgggacagttg

SEQ ID NO: 79 - DNA, 20 bp, Artificial Sequence
  Antisense Oligonucleotide
gtcattgtctccgggacagt

SEQ ID NO: 80 - DNA, 20 bp, Artificial Sequence
  Antisense Oligonucleotide
atgtcattgtctccgggaca

SEQ ID NO: 81 - DNA, 20 bp, Artificial Sequence
  Antisense Oligonucleotide
agatgtcattgtctccggga

SEQ ID NO: 82 - DNA, 20 bp, Artificial Sequence
  Antisense Oligonucleotide
aaagatgtcattgtctccgg

SEQ ID NO: 83 - DNA, 20 bp, Artificial Sequence
  Antisense Oligonucleotide
aaagatgtcattgtctccgg

SEQ ID NO: 84 - DNA, 20 bp, Artificial Sequence
  Antisense Oligonucleotide
agcgattcaaagatgtcatt

SEQ ID NO: 85 - DNA, 20 bp, Artificial Sequence
  Antisense Oligonucleotide
tgtagtacagcgattcaaag

SEQ ID NO: 86 - DNA, 20 bp, Artificial Sequence
  Antisense Oligonucleotide
cctgtagtacagcgattcaa

SEQ ID NO: 87 - DNA, 20 bp, Artificial Sequence
  Antisense Oligonucleotide
gtctccgtacatcttcctgt

SEQ ID NO: 88 - DNA, 20 bp, Artificial Sequence
  Antisense Oligonucleotide
catgccatgtctccgtacat

SEQ ID NO: 89 - DNA, 20 bp, Artificial Sequence
  Antisense Oligonucleotide
ttcatgccatgtctccgtac

SEQ ID NO: 90 - DNA, 20 bp, Artificial Sequence
  Antisense Oligonucleotide
gcttcatgccatgtctccgt

SEQ ID NO: 91 - DNA, 20 bp, Artificial Sequence
  Antisense Oligonucleotide
tggcttcatgccatgtctcc

SEQ ID NO: 92 - DNA, 20 bp, Artificial Sequence
  Antisense Oligonucleotide
tctggcttcatgccatgtct

SEQ ID NO: 93 - DNA, 20 bp, Artificial Sequence
  Antisense Oligonucleotide
tctctggcttcatgccatgt

SEQ ID NO: 94 - DNA, 20 bp, Artificial Sequence
  Antisense Oligonucleotide
acttgtgctactgaaatcat

SEQ ID NO: 95 - DNA, 20 bp, Artificial Sequence
  Antisense Oligonucleotide
taacttgtgctactgaaatc

SEQ ID NO: 96 - DNA, 20 bp, Artificial Sequence
  Antisense Oligonucleotide
tcccactgctcctaaagcca

SEQ ID NO: 97 - DNA, 20 bp, Artificial Sequence
  Antisense Oligonucleotide
cctcccactgctcctaaagc

SEQ ID NO: 98 - DNA, 20 bp, Artificial Sequence
  Antisense Oligonucleotide
accctcccactgctcctaaa

SEQ ID NO: 99 - DNA, 20 bp, Artificial Sequence
  Antisense Oligonucleotide
gtaccctcccactgctccta

SEQ ID NO: 100 - DNA, 20 bp, Artificial Sequence
  Antisense Oligonucleotide
tggtaccctcccactgctcc

SEQ ID NO: 101 - DNA, 20 bp, Artificial Sequence
  Antisense Oligonucleotide
gctggtaccctcccactgct

SEQ ID NO: 102 - DNA, 20 bp, Artificial Sequence
  Antisense Oligonucleotide
tctgctggtaccctcccact

SEQ ID NO: 103 - DNA, 20 bp, Artificial Sequence
  Antisense Oligonucleotide
tttctgctggtaccctccca

SEQ ID NO: 104 - DNA, 20 bp, Artificial Sequence
  Antisense Oligonucleotide
cctttctgctggtaccctcc

SEQ ID NO: 105 - DNA, 20 bp, Artificial Sequence
  Antisense Oligonucleotide
aacctttctgctggtaccct

SEQ ID NO: 106 - DNA, 20 bp, Artificial Sequence
  Antisense Oligonucleotide
agcaggcatattactcgtat

SEQ ID NO: 107 - DNA, 20 bp, Artificial Sequence
  Antisense Oligonucleotide
cagtgagcacgctaaaattt

SEQ ID NO: 108 - DNA, 20 bp, Artificial Sequence
  Antisense Oligonucleotide
gcaggtcagtgagcacgcta

SEQ ID NO: 109 - DNA, 20 bp, Artificial Sequence
  Antisense Oligonucleotide
aggcaggtcagtgagcacgc

SEQ ID NO: 110 - DNA, 20 bp, Artificial Sequence
  Antisense Oligonucleotide
acaggcaggtcagtgagcac

SEQ ID NO: 111 - DNA, 20 bp, Artificial Sequence
  Antisense Oligonucleotide
ctacaggcaggtcagtgagc

SEQ ID NO: 112 - DNA, 20 bp, Artificial Sequence
  Antisense Oligonucleotide
ggctacaggcaggtcagtga

SEQ ID NO: 113 - DNA, 20 bp, Artificial Sequence
  Antisense Oligonucleotide
ggggctacaggcaggtcagt

SEQ ID NO: 114 - DNA, 20 bp, Artificial Sequence
  Antisense Oligonucleotide
ctggggctacaggcaggtca

SEQ ID NO: 115 - DNA, 20 bp, Artificial Sequence
  Antisense Oligonucleotide
cactggggctacaggcaggt

SEQ ID NO: 116 - DNA, 20 bp, Artificial Sequence
  Antisense Oligonucleotide
agctgtcactggggctacag

SEQ ID NO: 117 - DNA, 20 bp, Artificial Sequence
  Antisense Oligonucleotide
cacatcctagctgtcactgg

SEQ ID NO: 118 - DNA, 20 bp, Artificial Sequence
  Antisense Oligonucleotide
tgcacatcctagctgtcact

SEQ ID NO: 119 - DNA, 20 bp, Artificial Sequence
  Antisense Oligonucleotide
gattcctgaacagtgtcatt

SEQ ID NO: 120 - DNA, 20 bp, Artificial Sequence
  Antisense Oligonucleotide
ccgattcctgaacagtgtca

SEQ ID NO: 121 - DNA, 20 bp, Artificial Sequence
  Antisense Oligonucleotide
attccgattcctgaacagtg

SEQ ID NO: 122 - DNA, 20 bp, Artificial Sequence
  Antisense Oligonucleotide
caggattccgattcctgaac

SEQ ID NO: 123 - DNA, 20 bp, Artificial Sequence
  Antisense Oligonucleotide
gacaggattccgattcctga

SEQ ID NO: 124 - DNA, 20 bp, Artificial Sequence
  Antisense Oligonucleotide
tcgacaggattccgattcct

SEQ ID NO: 125 - DNA, 20 bp, Artificial Sequence
  Antisense Oligonucleotide
aatcgacaggattccgattc

SEQ ID NO: 126 - DNA, 20 bp, Artificial Sequence
  Antisense Oligonucleotide
ctaatcgacaggattccgat

SEQ ID NO: 127 - DNA, 20 bp, Artificial Sequence
  Antisense Oligonucleotide
cagtctaatcgacaggattc

SEQ ID NO: 128 - DNA, 20 bp, Artificial Sequence
  Antisense Oligonucleotide
tccagtctaatcgacaggat

SEQ ID NO: 129 - DNA, 20 bp, Artificial Sequence
  Antisense Oligonucleotide
tgtccagtctaatcgacagg

SEQ ID NO: 130 - DNA, 20 bp, Artificial Sequence
  Antisense Oligonucleotide
gctgtccagtctaatcgaca

SEQ ID NO: 131 - DNA, 20 bp, Artificial Sequence
  Antisense Oligonucleotide
aagctgtccagtctaatcga

SEQ ID NO: 132 - DNA, 20 bp, Artificial Sequence
  Antisense Oligonucleotide
acaagctgtccagtctaatc

SEQ ID NO: 133 - DNA, 20 bp, Artificial Sequence
  Antisense Oligonucleotide
ccacaagctgtccagtctaa

SEQ ID NO: 134 - DNA, 20 bp, Artificial Sequence
  Antisense Oligonucleotide
tgccacaagctgtccagtct

SEQ ID NO: 135 - DNA, 20 bp, Artificial Sequence
  Antisense Oligonucleotide
cttgccacaagctgtccagt

SEQ ID NO: 136 - DNA, 20 bp, Artificial Sequence
  Antisense Oligonucleotide
cacttgccacaagctgtcca

SEQ ID NO: 137 - DNA, 20 bp, Artificial Sequence
  Antisense Oligonucleotide
aattcacttgccacaagctg

SEQ ID NO: 138 - DNA, 20 bp, Artificial Sequence
  Antisense Oligonucleotide
caaattcacttgccacaagc

SEQ ID NO: 139 - DNA, 20 bp, Artificial Sequence
  Antisense Oligonucleotide
ggcaaattcacttgccacaa

SEQ ID NO: 140 - DNA, 20 bp, Artificial Sequence
  Antisense Oligonucleotide
caggcaaattcacttgccac

SEQ ID NO: 141 - DNA, 20 bp, Artificial Sequence
  Antisense Oligonucleotide
tacaggcaaattcacttgcc

SEQ ID NO: 142 - DNA, 20 bp, Artificial Sequence
  Antisense Oligonucleotide
cactgatgcctcccctttgc

SEQ ID NO: 143 - DNA, 20 bp, Artificial Sequence
  Antisense Oligonucleotide
aacaaatgcttccaggtgaa

SEQ ID NO: 144 - RNA, 13 nt, Artificial Sequence
  siRNA
ugaagaauguuaa

SEQ ID NO: 145 - RNA, 14 nt, Artificial Sequence
  siRNA
agauagcaucuuaa

SEQ ID NO: 146 - RNA, 13 nt, Artificial Sequence
  siRNA
cugucgauuagaa

SEQ ID NO: 147 - RNA, 14 nt, Artificial Sequence
  siRNA
aacaagccagauua

SEQ ID NO: 148 - RNA, 13 nt, Artificial Sequence
  siRNA
agguagaauguaa

SEQ ID NO: 149 - RNA, 13 nt, Artificial Sequence
  siRNA
uguugagagugua

SEQ ID NO: 150 - RNA, 12 nt, Artificial Sequence
  siRNA
ugagagugugaa

SEQ ID NO: 151 - RNA, 13 nt, Artificial Sequence
  siRNA
agugugaccaaaa

SEQ ID NO: 152 - RNA, 13 nt, Artificial Sequence
  siRNA
gugugaccaaaaa

SEQ ID NO: 153 - RNA, 13 nt, Artificial Sequence
  siRNA
ugugaccaaaaga

SEQ ID NO: 154 - RNA, 13 nt, Artificial Sequence
  siRNA
gugaccaaaagua

SEQ ID NO: 155 - RNA, 13 nt, Artificial Sequence
  siRNA
gugaccaaaagua

SEQ ID NO: 156 - RNA, 13 nt, Artificial Sequence
  siRNA
ugcaccuuucuaa

SEQ ID NO: 157 - RNA, 13 nt, Artificial Sequence
  siRNA
gcaccuuucuaga

SEQ ID NO: 158 - RNA, 13 nt, Artificial Sequence
  siRNA
ccuuucuaguuga

SEQ ID NO: 159 - RNA, 13 nt, Artificial Sequence
  siRNA
gcaccuuucuaga

SEQ ID NO: 160 - RNA, 19 nt, Artificial Sequence
  siRNA
ucuagaaaggugcaaacau

SEQ ID NO: 161 - RNA, 14 nt, Artificial Sequence
  siRNA
uugcaccuuucuaa

SEQ ID NO: 162 - RNA, 20 nt, Artificial Sequence
  siRNA
uuagaaaggugcaaacaagg

SEQ ID NO: 163 - RNA, 19 nt, Artificial Sequence
  siRNA
gggccucuucugugacuuc

SEQ ID NO: 164 - RNA, 19 nt, Artificial Sequence
  siRNA
ccgacuggaagacacguuu

SEQ ID NO: 165 - RNA, 19 nt, Artificial Sequence
  siRNA
cccggguuaccaaugacaa

SEQ ID NO: 166 - RNA, 19 nt, Artificial Sequence
  siRNA
gggcaaaaagugcauccgu

SEQ ID NO: 167 - RNA, 25 nt, Artificial Sequence
  siRNA
uccaagccuaucaaguuugagcuuu

SEQ ID NO: 168 - RNA, 19 nt, Artificial Sequence
  siRNA
gccuaucaaguuugagcuu

SEQ ID NO: 169 - RNA, 25 nt, Artificial Sequence
  siRNA
gcaugaagacauaccgagcuaaauu

SEQ ID NO: 170 - RNA, 19 nt, Artificial Sequence
  siRNA
gcuaaauucuguggaguau

SEQ ID NO: 171 - RNA, 25 nt, Artificial Sequence
  siRNA
gccauuacaacugucccggagacaa

SEQ ID NO: 172 - RNA, 19 nt, Artificial Sequence
  siRNA
ggaagauguacggagacau

SEQ ID NO: 173 - RNA, 25 nt, Artificial Sequence
  siRNA
gagagugagagacauuaacucauua

SEQ ID NO: 174 - RNA, 25 nt, Artificial Sequence
  siRNA
gccaugucaaacaaauagucuaucu

SEQ ID NO: 175 - RNA, 19 nt, Artificial Sequence
  siRNA
ggguaccagcagaaagguu

SEQ ID NO: 176 - RNA, 19 nt, Artificial Sequence
  siRNA
ccagcagaaagguuaguau

SEQ ID NO: 177 - RNA, 19 nt, Artificial Sequence
  siRNA
gcagaaagguuaguaucau

SEQ ID NO: 178 - RNA, 19 nt, Artificial Sequence
  siRNA
gguuaguaucaucagauag

SEQ ID NO: 179 - RNA, 25 nt, Artificial Sequence
  siRNA
gagacugagucaaguuguuccuuaa

SEQ ID NO: 180 - RNA, 19 nt, Artificial Sequence
  siRNA
gcagacucagcucugacau

SEQ ID NO: 181 - RNA, 25 nt, Artificial Sequence
  siRNA
ucagcucugacauucugauucgaau

SEQ ID NO: 182 - RNA, 25 nt, Artificial Sequence
  siRNA
uccugucgauuagacuggacagcuu

SEQ ID NO: 183 - RNA, 19 nt, Artificial Sequence
  siRNA
gcuuguggcaagugaauuu